(19) United States
(12) Patent Application Publication
(10) Pub. No.: US 2024/0293612 A1
(43) Pub. Date: Sep. 5, 2024

(54) DRUG DELIVERY DEVICE

(71) Applicant: West Pharmaceutical Services, Inc., Exton, PA (US)

(72) Inventors: Michael SULLLIVAN, Birdsboro, PA (US); Lawton LAURENCE, Chester Springs, PA (US); Stefanie HUROWITZ, Exton, PA (US); John BASISTA, Exton, PA (US)

(21) Appl. No.: 18/574,754

(22) PCT Filed: Jul. 1, 2022
(86) PCT No.: PCT/US2022/035899

§ 371 Date: Dec. 28, 2023

Related U.S. Application Data
(60) Provisional application No. 63/217,312, filed on Jul. 1, 2021.
(60) Provisional application No. 63/235,117, filed on Aug. 19, 2021.
(60) Provisional application No. 63/252,401, filed on Oct. 5, 2021.

(51) Int. Cl. A61M 5/145; A61M 5/142; A61M 39/10

(52) U.S. Cl. CPC A61M 5/1456; A61M 5/14216; A61M 5/14566; A61M 2039/1005

(57) ABSTRACT

An infusion pump assembly (1) includes a housing (14) with a fluid reservoir (18) and an opening (22) to receive a connector (100) of an infusion set to fluidly connect to the reservoir. The assembly includes a switch assembly (70) that transitions between a connected state when the connector is inserted within the opening and a disconnected state when the connector is removed from the opening. An infusion set connects to the infusion pump. The infusion set can have a connector having a body including an outer wall that defines an outer perimeter and a wall cross-sectional shape that corresponds with a cross-sectional shape of an opening of the housing.

### CROSS REFERENCE TO RELATED APPLICATIONS

This application claims the benefit of U.S. Provisional Patent App. No. 63/217,312, filed Jul. 1, 2021, U.S. Provisional Patent App. No. 63/235,117, filed Aug. 19, 2021, and U.S. Provisional Patent App. No. 63/252,401, filed Oct. 5, 2021, the disclosures of which are hereby incorporated by reference herein.

### TECHNICAL FIELD

This disclosure generally relates to devices and methods of dispensing medicament to a user, and more particularly relates to an activation assembly for activating an infusion pump and an infusion set to be connectable with a compatible infusion pump.

### BACKGROUND

Controlled injections of medicament into a user's body, for example, in oncology treatments, requires a predetermined amount of the medicament to be supplied to the user. The medicament should be injected at the desired rate and for the necessary duration. Typically, the user must travel to a hospital or other clinical setting to receive such injections. Many users experience adverse effects after treatment and have difficulty returning from the hospital or clinic. Alternative arrangements allow for the user to administer medicament in a home setting either independently or with another person's assistance. However, most users, being laypeople, are not familiar with injection processes or with the proper protocol of preparing and/or administering the medicament. As such, there is a need for improved systems that can be easily utilized by untrained users to deliver medicament with decreased risk of improper connection of components or incorrect dosing or other injection processes.

The medicament can be delivered from a retainer via a pumping mechanism. The medicament can be transferred to the user at a desired location on the user's body. Patients who regularly receive such medicament deliveries may attach a portion of an infusion set to a skin surface at an approved location on their bodies through which the medicament can be injected. The infusion set then is then connected to the source of the medicament.

The pumping mechanism can be turned off prior to use and must be turned on before the medicament is administered. As such, there is a need for systems that allow a user, who is unfamiliar with medical components, to correctly turn on the pumping mechanism at the appropriate time and allow for the medicament to be administered to the desired location, at the right quantity, and without injuring the user or damaging components.

In addition, there is a further need for systems that allow a user, who is unfamiliar with medical components, to properly connect the medicament source with the infusion set so that the medicament can be administered to the desired location, at the right quantity, and without injuring the user or damaging components.

### SUMMARY

The foregoing needs are addressed by various aspects of infusion sets, infusion pumps, and infusion assemblies disclosed. According to an aspect of this disclosure, an infusion pump assembly includes a housing that has a fluid reservoir and that defines an opening configured to receive a connector of an infusion set to fluidly connect the infusion set to the reservoir. The infusion pump assembly further includes an actuator configured to drive fluid from the reservoir, a printed circuit board (PCB), a battery configured to selectively power the PCB and the actuator, and a switch assembly. The switch assembly includes a first switch and a second switch. The first switch is configured to initiate powering of the PCB by the battery. The first switch is configured to transition between an on state, in which the battery powers the PCB, and an off state, in which the battery does not power the PCB. The second switch is configured to transition between a connected state when the connector is inserted within the opening and a disconnected state when the connector is removed from the opening.

Optionally, the first switch and the second switch may be positioned at least partially within the opening of the housing. Optionally, the second switch may be configured to be biased to the disconnected state. Optionally, the assembly may include a spring configured to bias the second switch to the disconnected state.

Optionally, prior to insertion of the connector within the opening, the first switch may be in the off state and the second switch may be in the disconnected state, when the connector is inserted into the opening, the connector may transition the first switch to the on state and the second switch to the connected state, and if the connector is removed from the opening, the first switch may remain in the on state and the second switch may automatically transition to the disconnected state.

Optionally, the assembly may include a button configured to be manually actuated by a user, wherein actuation of the button activates the actuator only when the first switch is in the on state and the second switch is in the connected state. Optionally, when the motor is activated, the PCB may be configured to automatically deactivate the actuator upon the second switch transitioning from the connected state to the disconnected state.

Optionally, the assembly may include an indicator configured to indicate a status of the fluid delivery system, and a processor in electrical communication with the PCB. The processor may be configured to receive a first signal from the first switch and a second signal from the second switch, determine the status of the fluid delivery system based on the first signal and the second signal, and transmit a third signal to the indicator, the third signal being indicative of the status of the fluid delivery system.

Optionally, the status of the assembly may comprise at least one of an off status in which the first switch is in the off state and the second switch is in the disconnected state, an on status in which the first switch is in the on state and the second switch is in the connected state, and an error status in which the first switch is in the on state and the second switch is in the disconnected state.

Optionally, the indicator may include an audio indicator and a visual indicator configured to operate simultaneously to indicate the status of the fluid delivery system. Optionally, the connector may include an outer wall configured to contact both of the first switch and the second switch when the connector is inserted into the opening. Optionally, the connector may include a recess configured to receive the first switch therein, the recess being partly defined by a wall, and the wall being configured to contact the first switch. Optionally, the actuator may include a motor and a plunger rod assembly, wherein the motor is configured to move the plunger rod assembly to cause dispensing of the fluid from the fluid reservoir.

Optionally, the second switch and the connector may comprise corresponding electrical contacts, and wherein, when the connector is inserted into the opening, the corresponding electrical contacts become electrically connected and transition the second switch from the disconnected state to the connected state. Optionally, the first switch may be configured to transition from the off state to the on state and the second switch may be configured to transition from the disconnected state to the connected state during a single insertion of the connector into the opening. Optionally, the assembly may include an infusion set that includes the connector.

According to another aspect of this disclosure, a method of activating an infusion pump assembly includes receiving a first indication that a first switch has transitioned from an off state to an on state: receiving a second indication that a second switch has transitioned from a disconnected state to a connected state; and transmitting, in response to the reception of the first indication and the second indication, a status of the infusion pump assembly.

Optionally, the method may include indicating the status of the infusion pump assembly. Optionally indicating the status of the infusion pump assembly may include displaying the status of the infusion pump assembly. Optionally, receiving the first indication and receiving the second indication may be based on insertion of a connector of an infusion set into an opening of the infusion pump assembly.

Optionally, the method may include receiving a third indication that the second switch has transitioned from the connected state to the disconnected state when the connector of the infusion set is removed from the opening of the infusion pump assembly.

Optionally, the status of the infusion pump assembly may include at least one of an off status in which the first switch is in the off state and the second switch is in the disconnected state, an on status in which the first switch is in the on state and the second switch is in the connected state, and an error status in which the first switch is in the on state and the second switch is in the disconnected state. Optionally, the method may include activating a motor of the infusion pump assembly in response to the actuation of a button after reception of the first indication and the second indication.

According to an aspect of this disclosure, an infusion set, which is configured to connect to an infusion pump having a housing containing a fluid reservoir, may include a connector having a body that defines a channel at a proximal end of the body. The channel extends along a longitudinal axis. The body includes an outer wall extending distally and defining at least a portion of an outer perimeter of the body, wherein the outer wall defines a wall cross-sectional shape along a plane perpendicular to the axis, the cross-sectional shape configured to be asymmetrical along an axis perpendicular to the longitudinal axis and correspond with an opening cross-sectional shape of an opening of the housing. The body further includes a body retention member configured to engage a housing retention member of the housing to retain the body with the housing. The infusion set further includes a base configured to attach to an injection site and a flexible cannula extending from the proximal end of the body of the connector to the base and defining a fluid path from the connector to the base.

Optionally, the wall cross-sectional shape may be D-shaped.

Optionally, the wall cross-sectional shape may be substantially rotationally aligned with the opening cross-sectional shape, the body retention member being substantially rotationally aligned with the housing retention member.

Optionally, the connector may be rotationally fixed relative to the infusion pump when the connector is fully inserted within the opening of the housing.

Optionally, the connector may be rotationally fixed relative to the infusion pump when the connector is partially inserted within the opening of the housing.

Optionally, the body may include an inner wall spaced radially inward from the outer wall, wherein a distal end of the inner wall is positioned within the outer wall between the proximal end of the body and a distal end of the body opposite the proximal end.

Optionally, the outer wall may prevent rotation of the body of the connector when the connector is at least partially received within the opening of the housing.

Optionally, the body retention member may include a cantilevered arm configured to flex radially inward during insertion of the body through the opening of the housing and radially outward to engage the housing retention member to retain the body with the housing.

Optionally, the body retention member may be a first body retention member, and the body may include a second body retention member configured to engage a second housing retention member of the housing to further retain the body with the housing.

Optionally, the infusion set may include a needle positioned at least partially within the channel of the body, the needle extending distally from a distal end of the channel.

Optionally, the channel may extend about a channel axis, the channel axis being substantially parallel to a longitudinal axis of the body.

According to another aspect of the disclosure, a pump assembly includes an infusion set and a housing. The infusion set has a connector with a body that defines a channel at a proximal end of the body. The body further includes an outer wall extending distally and defining at least a portion of an outer perimeter of the body, wherein the outer wall defines a wall cross-sectional shape, and a body retention member. The housing includes a fluid reservoir. The housing defines an opening configured to receive at least a portion of the body of the connector. The housing further includes a housing retention member configured to engage the body retention member of the body to retain the body with the housing. An opening cross-sectional shape of the opening of the housing corresponds with the wall cross-sectional shape of the outer wall of the body.

Optionally, the connector may include a needle positioned at least partially within the channel of the body, the needle extending distally from a distal end of the channel.

Optionally, the fluid reservoir may include a septum, the needle being configured to pierce the septum when the body is at least partially positioned within the opening of the housing.

Optionally, the outer wall of the body may define a cavity, wherein the septum is received within the cavity when the body is at least partially inserted into the opening of the housing.

Optionally, the housing may include a plunger disposed within the fluid reservoir and a motor configured to drive the plunger through the fluid reservoir to drive fluid from the fluid reservoir through the needle of the connector.

Optionally, when the wall cross-sectional shape is substantially rotationally aligned with the opening cross-sectional shape, the body retention member may be substantially rotationally aligned with the housing retention member.

Optionally, the body retention member may be a first retention member, the housing retention member may be a first housing retention member, the body may include a second retention member, and the housing may include a second housing member configured to engage second body retention member of to further retain the body with the housing.

Optionally, engagement between the body retention member and the housing retention member may irreversibly couple the connector to the housing.

Optionally, the connector may be configured to be inserted into the opening along a linear trajectory such that the body of connector is rotationally immobile relative to the housing as it translates along the trajectory.

### BRIEF DESCRIPTION OF THE DRAWINGS

The present application is further understood when read in conjunction with the appended drawings. For the purpose of illustrating the subject matter, there are shown in the drawings exemplary aspects of the subject matter: however, the presently disclosed subject matter is not limited to the specific methods, devices, and systems disclosed. In the drawings:

FIG. 1 depicts a perspective view of an infusion pump assembly according to an aspect of this disclosure;

FIG. 2 depicts a schematic of an infusion pump assembly according to an aspect of this disclosure:

FIG. 3 depicts a perspective cross-sectional view of a portion of the infusion pump assembly of FIG. 1:

FIG. 4 depicts a cross-sectional view of an infusion pump according to an aspect of this disclosure with the first and second switches in their respective first configurations:

FIG. 5 depicts a cross-sectional view of the infusion pump of FIG. 4 with the first and second switches in their respective second configurations, and with a connector shown in phantom:

FIG. 6 depicts a perspective view of a first switch according to an aspect of this disclosure:

FIG. 7A depicts a top view of the first switch of FIG. 6, the first switch being in its first configuration:

FIG. 7B depicts a top view of the first switch of FIG. 6, the first switch being in its second configuration:

FIG. 8 depicts a perspective view of a connector according to an aspect of this disclosure:

FIG. 9A depicts a cross-sectional perspective view of an infusion pump assembly according to another aspect of the disclosure showing the connector engaged with the infusion pump and with the first switch;

FIG. 9B depicts a portion of the infusion pump assembly of FIG. 9A showing the connector relative to the first switch, the first switch being in its first configuration:

FIG. 9C depicts a portion of the infusion pump assembly of FIG. 9A showing the connector relative to the first switch, the first switch being in its second configuration:

FIG. 10A depicts a perspective view of a second switch according to an aspect of this disclosure:

FIG. 10B depicts a cross-sectional perspective view of the second switch of FIG. 10A:

FIG. 11A depicts a top view of the second switch of FIG. 10A shown in its first configuration according to an aspect of this disclosure;

FIG. 11B depicts a top view of the second switch of FIG. 10B shown in its second configuration according to an aspect of this disclosure:

FIG. 12 depicts a cross-sectional perspective view of an infusion pump assembly according to another aspect of the disclosure showing the connector engaged with the infusion pump and with the second switch:

FIG. 13A depicts a portion of the infusion pump assembly of FIG. 12 showing the connector relative to the second switch, the second switch being in its first configuration:

FIG. 13B depicts a portion of the infusion pump assembly of FIG. 12 showing the connector relative to the second switch, the second switch being in its second configuration;

FIG. 13C depicts a portion of an infusion pump assembly showing the connector relative to the second switch according to another aspect of the disclosure, the second switch being in its first configuration:

FIG. 13D depicts a portion of the infusion pump assembly of FIG. 13C with the second switch in its second configuration:

FIG. 13E depicts a cross-sectional view of a portion of an infusion pump assembly according to yet another aspect of this disclosure, showing the connector engaged with the housing:

FIG. 13F depicts a cross-sectional view of the infusion pump assembly of FIG. 15A, showing the connector disengaged from the housing; and

FIG. 14 depicts a cross-sectional perspective view of a portion of an infusion pump assembly according to another aspect of the disclosure.

FIG. 15 depicts an isometric view of an infusion pump according to an aspect of this disclosure:

FIG. 16 depicts a front plan view of the infusion pump of FIG. 15:

FIG. 17 depicts a cross-sectional view of a portion of the infusion pump of FIG. 15:

FIG. 18 depicts an angled perspective view of an infusion set according to an aspect of this disclosure:

FIG. 19 depicts an isometric view of a connector for an infusion set according to an aspect of this disclosure:

FIG. 20 depicts another angled perspective view of the connector of FIG. 19;

FIG. 21 depicts an angled cross-sectional view of the connector of FIG. 19;

FIG. 22 depicts another angled cross-sectional view of the connector of FIG. 19:

FIG. 23 depicts an angled cross-sectional view of the connector of FIG. 19 with a needle and a cannula according to an aspect of this disclosure:

FIG. 24A depicts a back plan view of the connector of FIG. 19:

FIG. 24B depicts a back plan view of a cross-sectional shape of the connector of FIG. 19 according to an aspect of this disclosure:

FIG. 24C depicts a back plan view of a cross-sectional shape of the connector of FIG. 19 according to another aspect of this disclosure; and

FIG. 25 depicts a perspective view of a connector adjacent an infusion pump before the connector is connected with the infusion pump.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

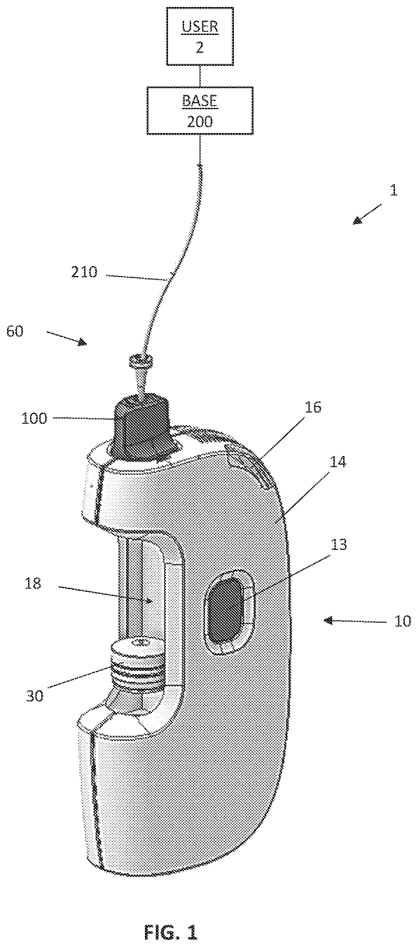

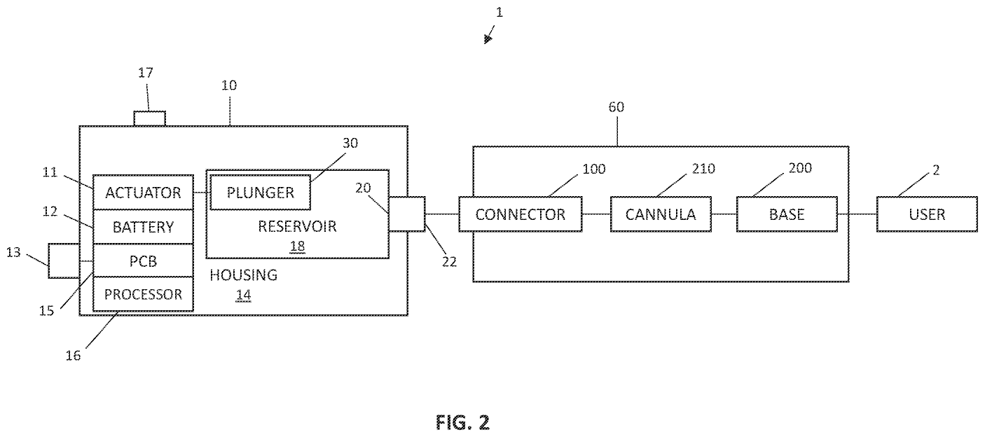

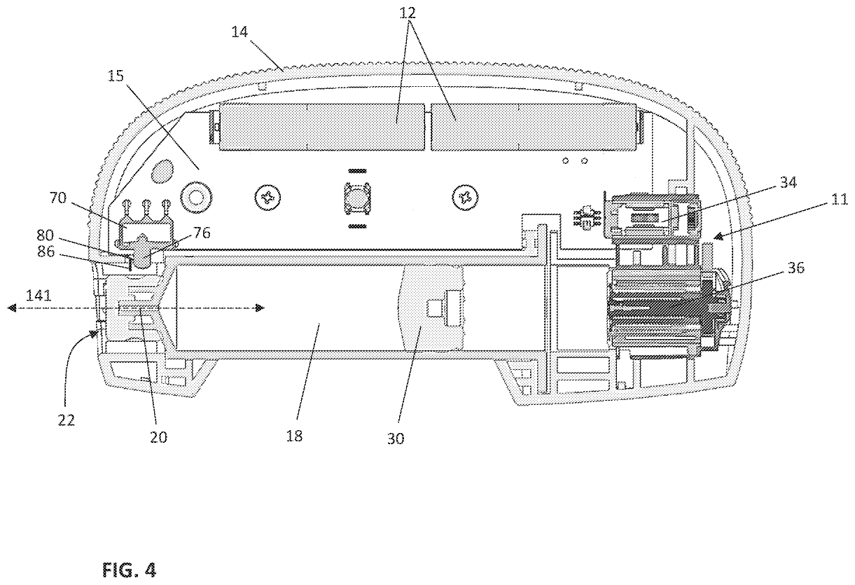

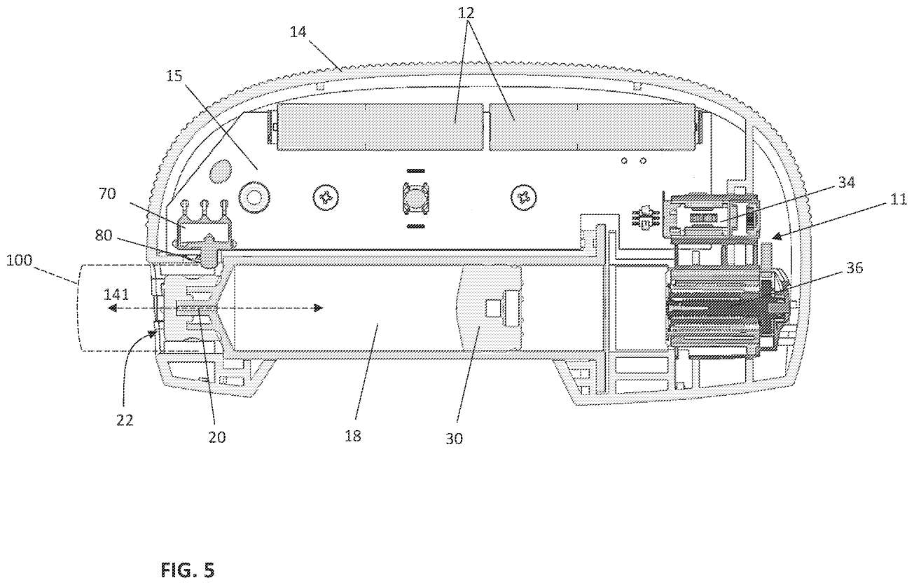

Referring to FIG. 1, an infusion pump assembly 1 is depicted. The infusion pump assembly 1 can include an infusion pump 10 and an infusion set 60 configured to connect to the infusion pump 10. The infusion set 60 is configured to connect to a user 2. The infusion pump 10 is shown having a housing 14 configured to receive a plurality of components therein. With reference to FIG. 2, a schematic is depicted showing components of an exemplary infusion pump assembly 1. It will be appreciated that other components may comprise the assembly, and that this disclosure is not limited by exclusion of additional components that may be present that are not otherwise depicted in FIG. 2. Referring to the schematic of FIG. 2 in combination with FIGS. 4 and 5, a reservoir 18 is disposed on or in the housing 14 and is configured to receive therein a liquid. The liquid may include one or more medicaments for treating a medical disease. A reservoir outlet 20 is defined at an end of the reservoir 18 and is configured to discharge the liquid from the reservoir 18 through the reservoir outlet 20.

A plunger 30 is movable within the reservoir 18. Movement of the plunger 30 through the reservoir 18 towards the reservoir outlet 20 can cause the liquid in the reservoir 18 to be expelled through the reservoir outlet 20. Movement of the plunger 30 may be actuated via an actuator 11, for example, an electronic motor 34 connected to a movable plunger rod 36 (shown in FIGS. 4 and 5). In some aspects, the plunger rod 36 may include a telescopic screw assembly. As depicted in FIG. 2, the actuator 11 may be operably connected to a power source 12 configured to provide power to the actuator 11 to cause movement of the plunger 30. In some aspects, the power source 12 may include one or more batteries.

The dispensing process can be operated by the user of the infusion pump assembly 1. The user can initiate the dispensing process by activating one or more components of the infusion pump 10 and placing the infusion pump 10 into a configuration from which dispensing can commence. In some aspects, the user can depress, toggle, or otherwise actuate a control interface 13 disposed on the housing 14. The control interface 13 can be a switch that is configured to be toggled by the user. As shown in FIG. 1, for example, the control interface 13 can be a button. The control interface 13 can be operably connected to a printed circuit board (PCB) 15 inside the housing 14 (see FIGS. 4 and 5), which can include electronic components connected thereto. A processor 16 is operably connected to the PCB 15 and is configured to receive and send signals through the PCB 15 from one or more components and to one or more components (see FIG. 2). The PCB 15 may include other electronic components, such as memory, connection ports, communication antennae, and the like, and may include instructions for operating one or more components of the infusion pump 10. The PCB 15 can be configured to receive power from the power source 12.

Dispensing of the liquid from the reservoir 18 can be controlled by the actuator 11. The actuator 11 is configured to be operated in response to instructions from the PCB 15. Activation of the actuator 11 can cause the plunger 30 to move within the reservoir 18 to cause the liquid therein to be dispensed. The rate and/or duration of dispensing may be determined by the processor 16 based on parameters of the reservoir 18, of the liquid in the reservoir 18, of the plunger 30, of the actuator 11, and/or of another component within, or operably connected with, the infusion pump 10. Alternatively, the rate and/or duration of dispensing can be preprogrammed into a memory of the PCB 15. In some aspects, the motor 34 can receive a signal from the PCB 15 and power from the power source 12 so as to cause movement of the plunger rod 36. The plunger rod 36 contacts the plunger 30 and can move the plunger 30 within the reservoir 18, thus moving the liquid therein towards and out through the reservoir outlet 20.

As shown in FIGS. 1-3, the infusion pump 10 may be connected with an infusion set 60. The infusion set 60 is configured to receive the liquid from the infusion pump 10 and to transfer the liquid to the desired injection site.

As shown in the exemplary embodiments of FIGS. 1-3, the infusion set 60 can include a connector 100 that is configured to connect to the housing 14 of the infusion pump 10. When the infusion set 60 is connected to the infusion pump 10, the infusion pump 10 can be operated to expel the liquid in the reservoir 18 out of the reservoir 18 through the outlet 20 and into the infusion set 60. The liquid can then travel through the infusion set 60 to the desired target site. The target site may be an injection site on the body of a user. In some aspects, the connection of the connector 100 may cause activation of one or more components of the infusion pump 10 described above and/or can cause actuation of the one or more components, as will be described in detail below.

The infusion set 60 may be connectable to the infusion pump 10 prior to liquid in the reservoir 18 being discharged. In some aspects, the infusion set 60 may be intended to be permanently affixed to the infusion pump 10, such that, after being connected to the infusion pump 10, the infusion set 60 is not designed or intended to be separated from the infusion pump 10. It will be appreciated that separation in such embodiments may physically be achieved by utilizing tools and/or excessive force, but that such separation would go beyond the intended interaction of components and their respective uses.

Referring to FIGS. 4-5, a cross-sectional view of the infusion pump 10 is depicted showing, inter alia, the housing 14 that can encompass therein one or more of the described components of the infusion pump 10. An opening 22 is defined on, or in, the housing 14. The opening 22 is configured to define, receive therein, or otherwise be adjacent or in communication with the reservoir outlet 20 of the reservoir 18. The opening 22 is configured to receive therein, or thereon, the connector 100. During operation, the medicament in the reservoir 18 can be moved towards and through the reservoir outlet 20 and into and through the connector 100. The connector 100 (shown in phantom in FIG. 5) can be affixed or otherwise secured in a desired position and orientation relative to the opening 22 of the housing 14.

The infusion pump 10 includes a first switch 70 configured to be actuated by the user to cause the infusion pump 10 to turn on. When the first switch 70 is actuated, the PCB 15 is configured to receive power from the power source 12 (e.g., one or more batteries) and to supply power and operating signals and instructions to the actuator 11. The first switch 70 has a first configuration, in which the first switch 70 is in the “off” position, and where the PCB 15 is not receiving power from the power source 12. The first switch 70 has a second configuration, in which the first switch 70 is in the “on” position, where the PCB 15 can receive power from the power source 12. In operation, the first switch 70 can be moved from the first configuration to the second configuration prior to use. This allows the infusion pump 10 to be generally in the “off” position and not supply power from the power source 12 to the PCB 15 or any of the other components until it is necessary to do so. This prevents inadvertent discharge of the power source 12. This further allows for the power source 12 to have a smaller capacity that is sufficient to operate the infusion pump 10 during use without requiring excess capacity to power the infusion pump 10 before the first switch 70 is moved into the second configuration.

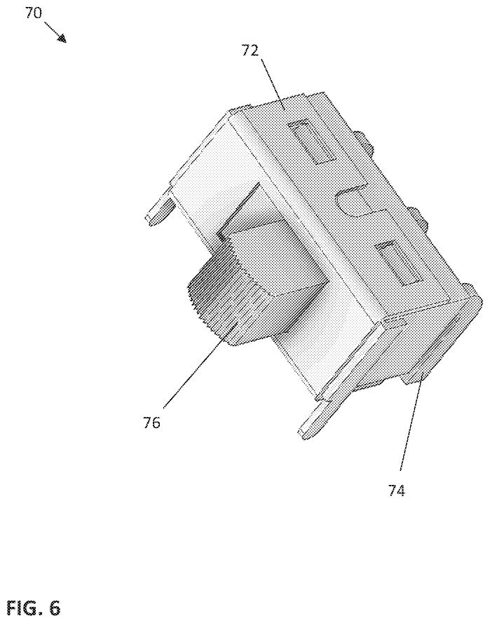

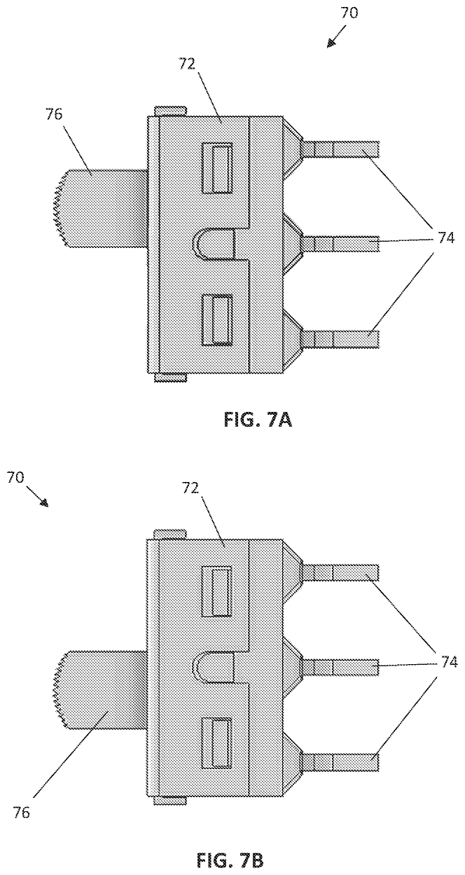

The first switch 70 can include a button, lever, rocker, slide, or another suitable mechanical switching mechanism. In some aspects, the first switch 70 may include a sliding switch, in which one component moves relative to another component to selectively form or remove an electrical connection. Referring to FIGS. 6, 7A, and 7B, the first switch 70) can include a body 72 that can be attached to the infusion pump 10. The body 72 can be connected to the PCB 15 via one or more connectors 74. Electrical signals can travel via the one or more connectors 74 from the first switch 70 to the PCB 15 or vice versa. A movable arm 76 is disposed on or in the body 72. The arm 76 is configured to move between a first position and a second position spaced from the first position. Referring specifically to FIG. 7A, when the arm 76 is in the first position, the first switch 70 is in the first configuration (i.e., the “off” configuration). Referring to FIG. 7B, when the arm 76 is in the second position, the first switch 70 is in the second configuration (i.e., the “on” configuration). When the arm 76 is in the second position, the first switch 70 is configured to cause the PCB 15 to receive power from the power source 12.

The first switch 70 may be actuated from the first configuration to the second configuration by the connector 100. For example, when the connector 100 is moved into, or onto, the opening 22 of the housing 14, the connector 100 can toggle the first switch 70) from the first configuration into the second configuration.

As shown in FIGS. 4-5, the arm 76 may extend into the opening 22. When the connector 100 is connected to the housing 14 of the infusion pump 10, the connector 100 may contact the arm 76 of the first switch 70. During movement of the connector 100 relative to the housing 14, the connector 100 can push the arm 76 of the first switch 70 from the first position into the second position, thus toggling the first switch 70 from the first (“off”) configuration to the second (“on”) configuration. Movement of the connector 100 relative to the housing 14 can be along an insertion axis 141 shown in the figures. The first position of the arm 76 may be spaced from the second position in a direction along the insertion axis 141. Referring to FIG. 5, a connector 100 is shown in phantom in contact with the arm 76 of the first switch 70, the arm 76 having been moved by the connector 100 from its first position to its second position. Thus, FIG. 4 depicts the first switch 70 in the first configuration, and FIG. 5 depicts the first switch 70 in the second configuration.

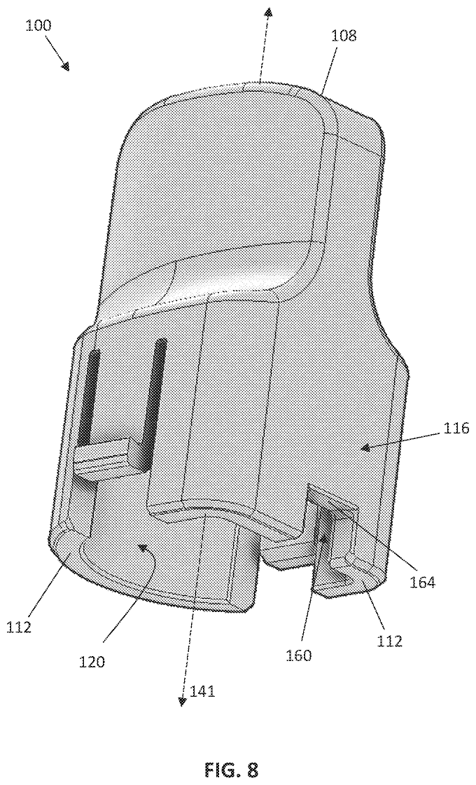

In some aspects, the connector 100 may include various features configured to engage with the first switch 70. Referring to FIG. 8, an exemplary connector 100 is depicted having a proximal end 108 and a distal end 112 spaced from the proximal end 108 along, for example, the insertion axis 141. The connector 100 includes an inner wall or inner surface 120, which defines a cavity inside the connector 100, and an outer wall or outer surface 116 spaced from the inner surface 120 in a radial direction, for example, in a direction away from the insertion axis 141. In some aspects, the connector 100 may include a recess 160 defined at least partially on the outer surface 116. In some aspects, such as shown in FIG. 8, the recess 160 may extend through the connector 100 between the outer surface 116 and the inner surface 120. The recess 160 is partially defined by a wall 164. In some aspects, the wall 164 may be substantially orthogonal to the insertion axis 141 when the connector 100 is being connected to, or is connected to, the infusion pump 10.

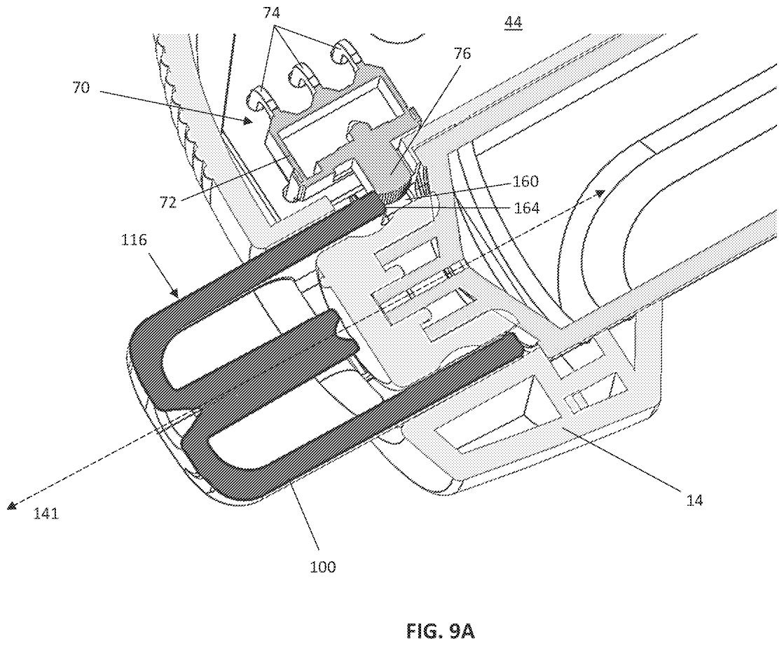

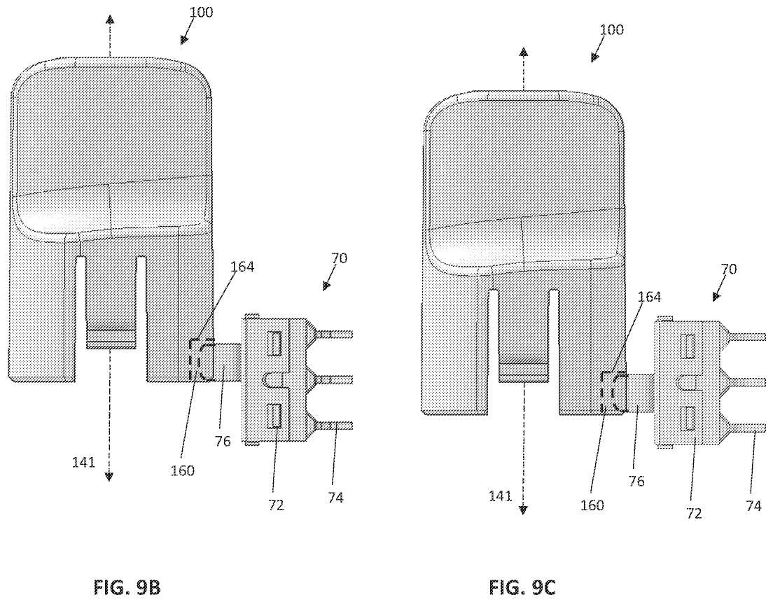

As the connector 100 is connected to the infusion pump 10, the connector 100 is moved along the insertion axis 141 towards the opening 22 on the housing 14. Referring to FIG. 9A, a cross-sectional view depicts the connector 100 connected to the housing 14 of the infusion pump 10. During connection, the arm 76 of the first switch 70 can be received into the recess 160 defined on the connector 100. As the connector 100 is moved towards the housing 14, the wall 164 can contact the arm 76 and cause movement of the arm 76 from the first position to the second position. The specific position of the recess 160, the wall 164, and the arm 76 can be predetermined such that the arm 76 is moved fully into the second position substantially at the same time as the connector 100 is fully connected to the infusion pump 10. Thus, the first switch 70 can be moved into the second (“on”) configuration when the connector 100 is fully connected to the infusion pump 10. This can prevent inadvertent activation of the components of the infusion pump 10 if the connector 100 is improperly connected, not fully connected, or partially connected and then disconnected (e.g., if the user changes his/her mind about commencing with the injection process). Referring to FIGS. 9B and 9C, the connector 100 is shown relative to the first switch 70. FIG. 9B shows the first switch 70 in the first configuration, with the arm 76 being received into the recess 160 (shown in phantom) without the wall 164 having yet pushed the arm 76 into the second position. FIG. 9C shows the connector 100 moved further towards the housing (not shown in FIGS. 9B and 9C) with the wall 164 of the recess 160 contacting the arm 76 of the first switch 70 and pushing the arm 76 into the second position. The first switch 70 is in the first (“off”) configuration in FIG. 9B and in the second (“on”) configuration in FIG. 9C.

After the connector 100 is fully engaged with the infusion pump 10 and the first switch 70 is in the second configuration, the PCB 15 is configured to receive power from the power source 12, and the actuator 11 can be caused to dispense the medicament (e.g., by actuating the motor 34 to move the plunger rod 36). When the first switch 70 is in the second configuration, the user can initiate dispensing of medicament by actuating the control interface 13. In some aspects, the control interface 13 can be a push button, and the actuation process can include the user pushing the button (shown in FIG. 1). Actuation of the control interface 13 can send a signal to or through the PCB 15 to the actuator 11 to initiate dispensing of the medicament from the reservoir 18. If the control interface 13 is actuated when the first switch 70 is in the first (“off”) configuration, no signal is sent to or through the PCB 15, and thus such actuation while the first switch 70 is in the first configuration would not initiate dispensing of the medicament.

The first switch 70 may be configured such that it can be moved from the first configuration to the second configuration when the connector 100 is connected to the infusion pump 10 and to remain in the second configuration if or when the connector 100 is disconnected from the infusion pump 10.

The infusion pump 10 may include a second switch 80 configured to be actuated by the user to allow dispensing of the medicament. The second switch 80 can communicate with the PCB 15 to indicate to the PCB 15 whether the connector 100 is properly connected to the infusion pump 10 or, alternatively, whether the connector 100 is improperly connected to, or has been disconnected from, the infusion pump 10. The second switch 80 has a first configuration, in which the second switch 80 is in the “off” position, and a second configuration, in which the second switch 80 is in the “on” position. The first configuration can also be referred to as a disconnected state, and the second configuration can also be referred to as a connected state. The second switch 80 can be moved from the first configuration to the second configuration or vice versa. In some aspects, the second switch 80 can be biased into the first configuration. The second switch 80 can be moved from the first configuration to the second configuration by the connector 100, as will be described further below:

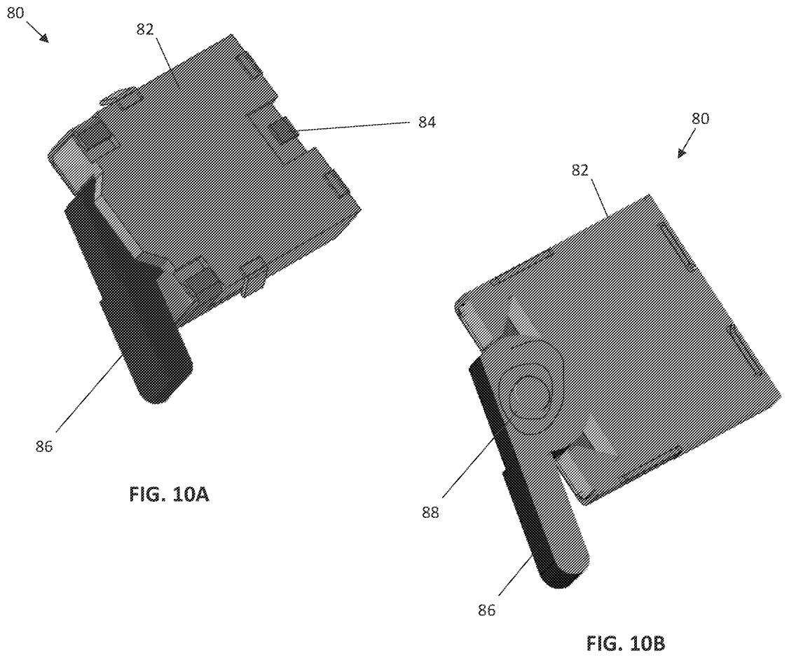

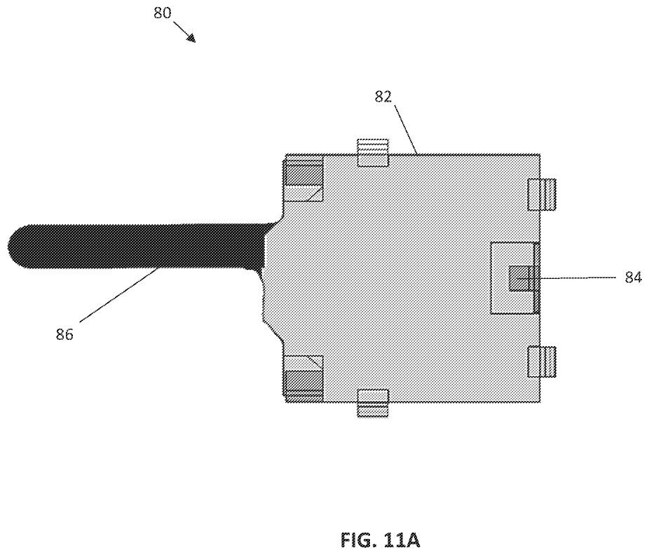

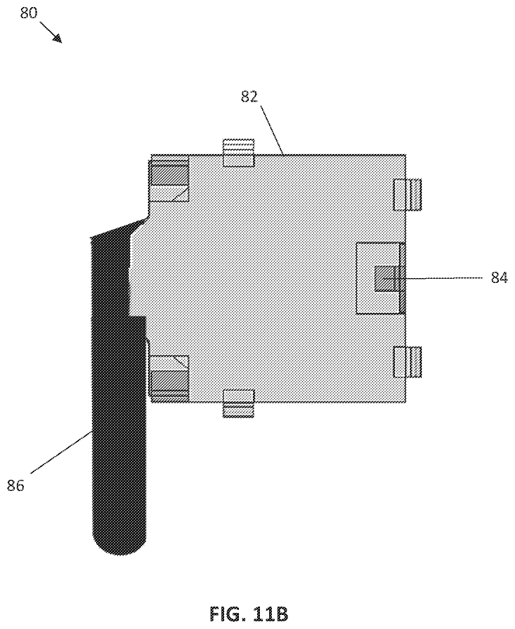

The second switch 80 can include a button, lever, rocker, slide, or another suitable mechanical switching mechanism. In some aspects, the second switch 80 may include a lever that can be pivoted towards or away from another component to selectively form or remove an electrical connection, respectively. Referring to FIGS. 10A-11B, the second switch 80 may include a body 82 that can be attached to the infusion pump 10. The body 82 can be connected to the PCB 15 via one or more connectors 84. Electrical signals can travel via the one or more connectors 84 from the second switch 80 to the PCB 15 or vice versa. A movable arm 86 is disposed on or in the body 82. The arm 86 is configured to move between a first position and a second position spaced from the first position. When the arm 86 is in the first position, the second switch 80 is in the first configuration (i.e., the “off” configuration) (see FIG. 11A). When the arm 86 is in the second position, the second switch 80) is in the second configuration (i.e., the “on” configuration) (see FIG. 11B). The arm 86 may be moved from the first position to the second position by an application of a force thereon to cause the arm 86 to pivot from the first position to the second position. The arm 86 may be biased into the first position, such that when the force applied to the arm 86 is removed or sufficiently reduced, the arm 86 moves back from the second position into the first position. The second switch 80 may include a biasing element 88 (see FIG. 10B) configured to bias the arm 86 in the first position. The biasing element 88 may include a spring.

When the second switch 80 is in the first configuration, a first signal can be sent to or through the PCB 15. When the second switch 80 is in the second configuration, a second signal different from the first signal can be sent to or through the PCB 15. The first and second signals can indicate operation parameters of the infusion pump 10. For example, the first signal can include an indication that the connector 100 is not fully engaged with the housing 14. In some aspects, the first signal can comprise an indication that the connector 100 is improperly engaged with the housing 14. The second signal can comprise an indication that the connector 100 is fully and/or properly engaged with the housing 14.

The second switch 80 may be actuated from the first configuration to the second configuration by the connector 100. For example, when the connector 100 is moved into, or onto, the opening 22 of the housing 14, the connector 100 can toggle the second switch 80 from the first configuration into the second configuration.

Referring again to FIGS. 4 and 5, the arm 86 of the second switch 80 may extend into the opening 22. When the connector 100 is connected to the housing 14 of the infusion pump 10, the connector 100 may contact the arm 86 of the second switch 80. During movement of the connector 100 relative to the housing 14, the connector 100 can push the arm 86 of the second switch 80 from the first position into the second position, thus toggling the second switch 80 from the first (“off”) configuration to the second (“on”) configuration. Referring specifically to FIG. 5, a connector 100 is shown in phantom in contact with the arm 86 of the second switch 80, the arm 86 having been moved by the connector 100 to its second position. Thus, FIG. 4 depicts the second switch 80 in the first configuration, and FIG. 5 depicts the second switch 80 in the second configuration. As the connector 100 contacts the arm 86, the connector 100 applies a force to the arm 86. It will be understood that the force applied by the connector 100 should be sufficient to cause the arm 86 to move from the first position to the second position and to overcome a responsive biasing force applied toward the first position by the biasing element 88.

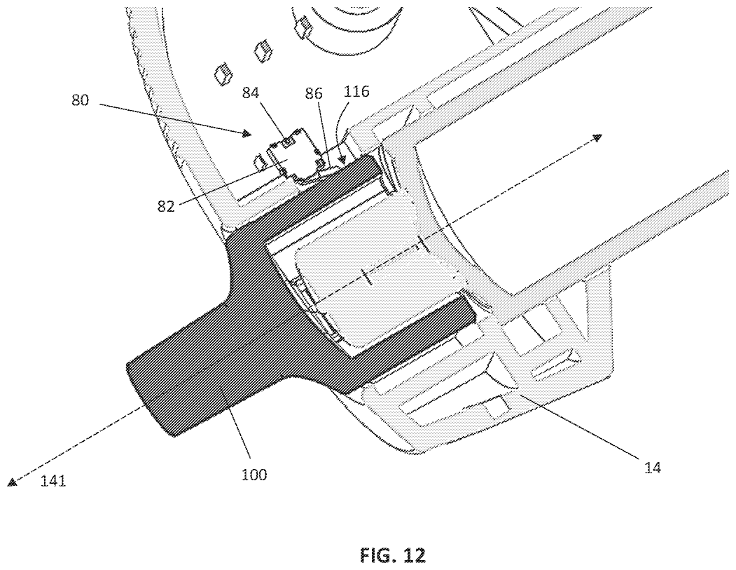

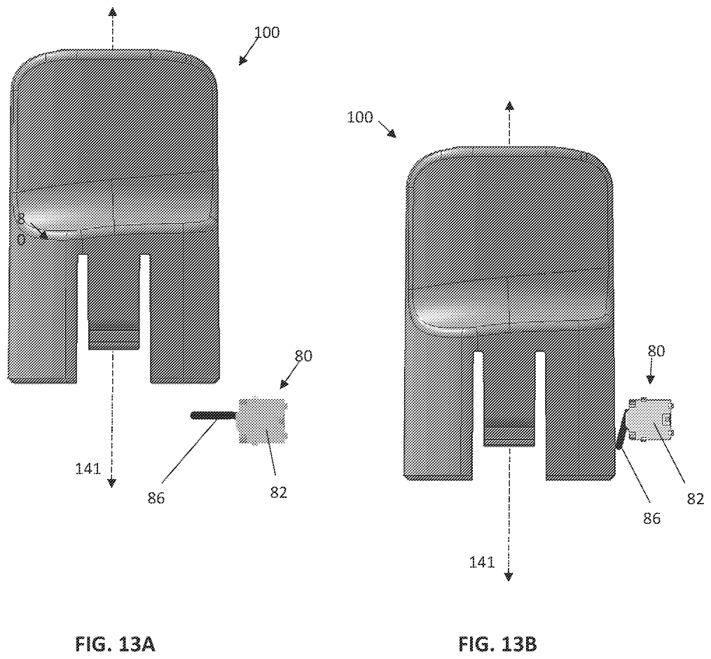

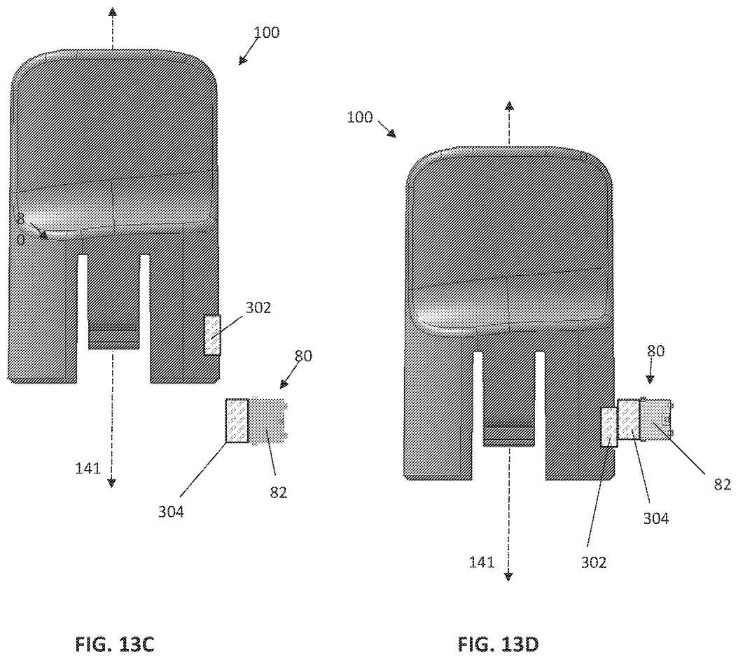

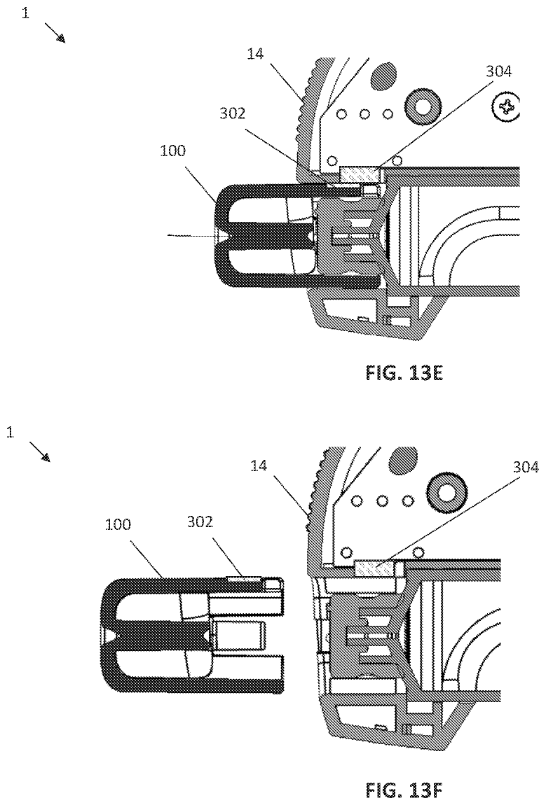

In some aspects, the outer surface 116 of the connector 100 can contact the arm 86 of the second switch 80 during connection of the connector 100. The outer surface 116 can be configured to slidably engage the arm 86 and push the arm 86 from the first position to the second position. Referring to FIG. 12, a cross-sectional view depicts the connector 100 connected to the housing 14 of the infusion pump 10. While the connector 100 is engaged with the housing 14, the arm 86 of the second switch 80 can remain in contact with the outer surface 116 of the connector. During such engagement, the second switch 80 is in the second (“on”) configuration. Referring to FIGS. 13A and 13B, the connector 100 is shown relative to the second switch 80. FIG. 13A shows the second switch 80 in the first configuration, with the arm 86 being spaced from the outer surface 116 of the connector 100. FIG. 13B shows the connector 100 moved further towards the housing (not shown in FIGS. 13A and 13B) with the outer surface 116 of the connector 100 contacting the arm 86 of the second switch 80 and pushing the arm 86 into the second position. The second switch 80 is in the first (“off”) configuration in FIG. 13A and in the second (“on”) configuration in FIG. 13B.

If, or when, the connector 100 is disengaged from the housing 14, the outer surface 116 can be moved away from the second switch 80 such that the biasing force applied to the arm 86 by the biasing element 88 can move the arm 86 into the first position. If the connector 100 is engaged with the housing 14 but is improperly oriented or angled relative to the housing 14, the outer surface 116 can be spaced away from the second switch 80 so as to allow the arm 86 to be moved into, or remain in, the first position. It will be appreciated that the shape and dimensions of the connector 100 and the housing 14 can be predetermined such that, when the connector 100 is fully and properly engaged with the housing 14, the distance between the outer surface 116 of the connector 100 and the second switch 80 is sufficient to cause the outer surface 116 to move and retain the arm 86 in the second position, and, when the connector 100 is not fully engaged or is improperly engaged with the housing 14, the distance between the outer surface 116 and the second switch 80 is sufficient to allow the arm 86 to either remain in the first position or to be moved into the first position by the biasing element 88.

In some aspects, the engagement between the connector 100 and the housing 14 can be detected and/or quantified by an electronic connection established between the connector 100 and the housing 14. Referring to FIGS. 13C-13F, an electronic connection may be formed between the second switch 80 and a transmitter 302 on the connector 100. The second switch 80 may include a receiver 304 configured to establish an electrical connection with the transmitter 302. In such aspects, the second switch 80 may be without the arm 86 and the biasing element 88 described above. The transmitter 302 can be disposed on or in the connector 100, for example, on, or adjacent to, the outer surface 116 of the connector 100. The receiver 304 can be disposed on, or within, the second switch 80. In some aspects, the receiver 304 may be disposed elsewhere on the housing 14.

The receiver 304 can be configured to send and/or receive a signal to and/or from the transmitter 302 on the connector 100. A signal can be sent and/or received between the transmitter 302 and the receiver 304 when the connector 100 is at a desired location relative to the housing 14, for example, when the connector 100 is fully moved into the opening 22 towards the housing 14 along the insertion axis 141. In some aspects, one or more signals can be sent and/or received based on the relative positioning of the connector 100 with respect to the housing 14. A signal can be sent and/or received based on the distance of the transmitter 302 on the connector 100 from the receiver 304 on the housing 14. The signal or signals can be indicative of a characteristic of the position of the connector 100 relative to the housing 14, and specifically relative to the opening 22. It should be further appreciated that the above arrangement can be reversed, such that the transmitter 302 is disposed on the housing 14 while the receiver 304 is disposed on the connector 100.

The transmitter 302 and the receiver 304 may be any suitable electronic transmitter and receiver and may utilize any suitable transmission and connection mechanisms, such as, for example, electronic contacts, magnets, radio frequency identification (RFID), or another suitable mechanism. In some aspects, the transmitter 302 may include a first electronic contact or lead, and the receiver 304 may include a second electronic contact or lead. An electronic connection can be established between the connector 100 and the housing 14 when the first electronic contact or lead of the connector 100 contacts the second electronic contact or lead of the housing 14. In some aspects, the transmitter 302 may include a magnet, and the receiver 304 may include a magnetic element that is configured to be affected by the magnet. Movement of the connector 100 relative to the housing 14 can cause the magnetic element to move by a known distance or in a known direction, which can be indicative of the relative position between the connector 100 and the housing 14. It should be appreciated that the above arrangement can be reversed, such that the receiver 304 may include the magnet while the transmitter 302 may include the magnetic element. In some aspects, the transmitter 302 may include a passive RFID transponder, and the housing 14 may include an RFID reader or scanner. In some aspects, the connector 100 may include an active RFID transponder, and the housing 14 may include an RFID reader or scanner.

The signals associated with the electronic connection between the transmitter 302 and the receiver 304 can be transmitted to the processor 16. The signals can be indicative of a parameter associated with the positioning and/or orientation of the connector 100 relative to the housing 14. The processor 16 can perform a calculation or analysis based on the signals between the transmitter 302 and the receiver 304. The analysis or calculation can be based on an operating program or instructions provided to the processor 16 by a user or stored in the memory on the PCB 15. The processor 16 may cause an action to occur based on the analyzed signals. Suitable actions can include permitting actuation of the infusion pump 10, actuating the motor 34, precluding actuation of the infusion pump 10 and/or the motor 34, storing data within the memory of the PCB 15, transmitting data to an external computing device, and/or notifying a user of a condition of the infusion pump assembly 1 (e.g., generating an auditory, visual, and/or tactile notification).

The connection established between the transmitter 302 and the receiver 304 may serve a similar purpose as the mechanical movement of the arm 86 by the connector 100. A signal sent and/or received between the transmitter 302 and the receiver 304 may be associated with the position of the connector 100 relative to the housing 14. The processor 16 can analyze this signal to determine if the connector 100 is properly secured and/or oriented within the opening 22. The processor 16 can utilize this signal to determine if the connector 100 is improperly connected to, or has been disconnected from, the infusion pump 10.

In some aspects, the second switch 80 may include both, the mechanical arm 86 and biasing element 88 arrangement and the transmitter 302 and receiver 304 arrangement described above.

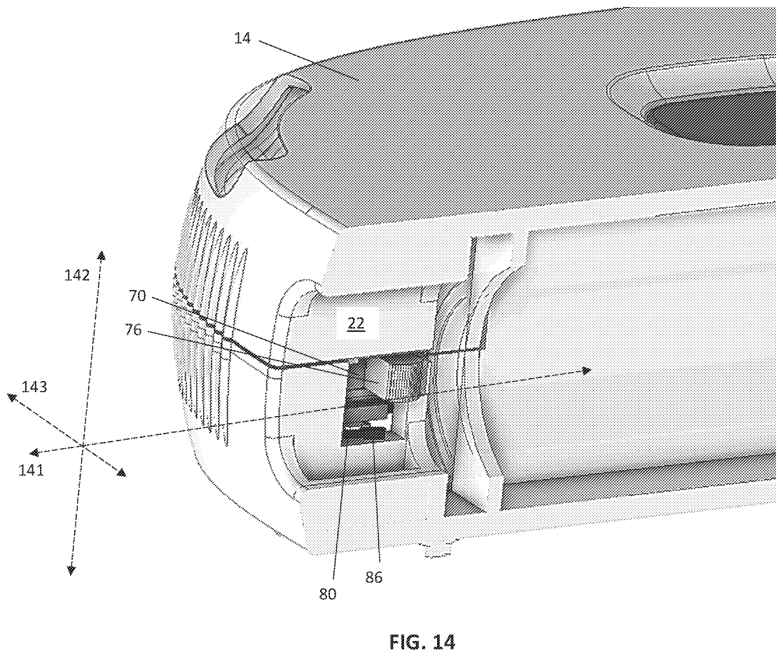

The first switch 70 and the second switch 80 may be arranged adjacent to each other so that the connector 100 can contact both the first and second switches 70 and 80 when the connector 100 is inserted into the opening 22 of the housing 14. Referring to FIG. 14, a cross-sectional view depicts the first switch 70 and the second switch 80 arranged in the housing 14. The arms 76 and 86 of the first and second switches 70 and 80, respectively, are shown extending, at least partially, into the opening 22. The first switch 70 may be offset from the second switch 80 along a transverse axis 142 that is orthogonal to the insertion axis 141. In some aspects, the first switch 70 may be offset from the second switch 80 along a lateral axis 143 that is orthogonal to the insertion axis 141 and the transverse axis 142. In some aspects, the first switch 70 may be offset from the second switch 80 along the insertion axis 141. In some aspects, the first switch 70 may be rotationally offset about one or more of the insertion axis 141, transverse axis 142, and/or lateral axis 143 relative to the second switch 80. It will be appreciated that regardless of relative positioning of the first switch 70 to the second switch 80, the respective arms 76 and 86 may extend into the opening 22 and be configured to be contacted by the connector 100 when the connector 100 is moved toward the housing 14.

Such an arrangement enables an effective feedback loop to and from the opening 22 of the housing 14 to the PCB 15. In the event of accidental disconnection of the connector 100, the second switch 80 is moved into, or remains in, the first configuration. When the second switch 80 is in the first configuration, a first signal can be sent to or through the PCB to indicate that the connector 100 is not connected or is improperly connected. This signal can be used to communicate an error designation to the user. This can alert the user that dispensing of the medicament either cannot commence or that dispensing could be improper. This can also alert the user to possible leaks due to disconnected or improperly connected components. Such undesirable arrangement can lead to missed doses or decreased amounts of dispensed medicament.

During use, when the connector 100 is connected to the housing 14 in the desired proper manner, the first switch 70 is placed into its second configuration, thus allowing the PCB 15 and the other electronic components to receive power from the power source 12, and the second switch 80 is placed into its second configuration, indicating to the PCB 15 and/or the processor 16 that the connector 100 is properly connected. To initiate dispensing of the medicament from the reservoir 18, the user can actuate the control interface 13 (e.g., by pushing the button shown in FIG. 1). When the button 13 is pushed, the processor 16 receives the signal from the button 13 and relays a signal through the PCB to the actuator 11 to instruct the actuator 11 to begin dispensing medicament. The motor 34 of the actuator 11 begins to operate and causes the connected plunger rod 36 to move the plunger 30 through the reservoir 18 towards the reservoir outlet 20, thus displacing the medicament in the reservoir 18 and forcing the medicament out of the reservoir 18, through the reservoir outlet 20, and into and through the connector 100.

If the connector 100 is disconnected from the infusion pump 10 or is otherwise moved into a non-desirable position or orientation relative to the infusion pump 10, the second switch can transition from the second position to the first position as described above. When this happens, the PCB 15 (e.g., the processor 16 on the PCB 15) may send a signal to the actuator 11 to stop actuation of the medicament. This can include stopping operation of the motor 34.

In some aspects, the infusion pump 10 may include one or more indicators 17 configured to alert the user of a status of the infusion pump 10. The indicators 17 may include visual indicators, auditory indicators, tactile indicators, or combinations of the above. As shown in FIG. 1, for example, the infusion pump 10 may include a visual indicator 17 configured to provide a visible indication to the user. The visual indicator 17 may include a light-emitting diode configured to selectively turn on or off in response to one or more statuses of the infusion pump 10. The visual indicator 17 may include a plurality of light-emitting diodes. In some aspects, the visual indicator 17 may emanate one or more colors that can be associated with one or more statuses of the infusion pump 10. In some aspects, the visual indicator 17 may operate in an on/off pattern to indicate one or more statuses, such as fully on, fully off, and/or intermittent switching between on and off (i.e., blinking). In some aspects, the infusion pump 10 may include an auditory indicator 17 configured to produce a sound to alert the user of a status of the infusion pump 10. The auditory indicator 17 can produce one or a plurality of different sounds and/or produce sounds in one or more patterns of sounds to indicate different statuses. In some aspects, the infusion pump 10 may include a plurality of indicators 17, for example a visual indicator 17 and an auditory indicator 17. The one or more indicators 17 may be operably connected to the PCB 15 and may be controlled by one or more components of the PCB 15 (e.g., the processor 16).

The PCB 15 can send different signals to the one or more indicators 17 to alert the user to one or more statuses of the infusion pump 10. For example, when the connector 100 is properly connected to the housing 14, the PCB 15 may send a first signal to indicate that the infusion pump 10 has been turned on. When the user initiates the dispensing processes by actuating the control interface 13 (e.g., by pushing a button), the PCB 15 may send a second signal different from the first signal to indicate to the user that dispensing is initiated. If the connector 100 becomes disconnected or otherwise moved to an undesired position such that the second switch is moved back to the first position, the PCB 15 can send a third signal different from the first and second signals to indicate to the user that the connector 100 is disconnected. It should be understood that the infusion pump assembly 1 can be configured to provide a plurality of different indicators to the user. The infusion pump assembly 1 can alert the user to one or more “error” statuses if the connector 100 has been disconnected or otherwise moved to an undesired position after the first switch 70 has been moved to the second configuration and that infusion pump 10 has been turned on.

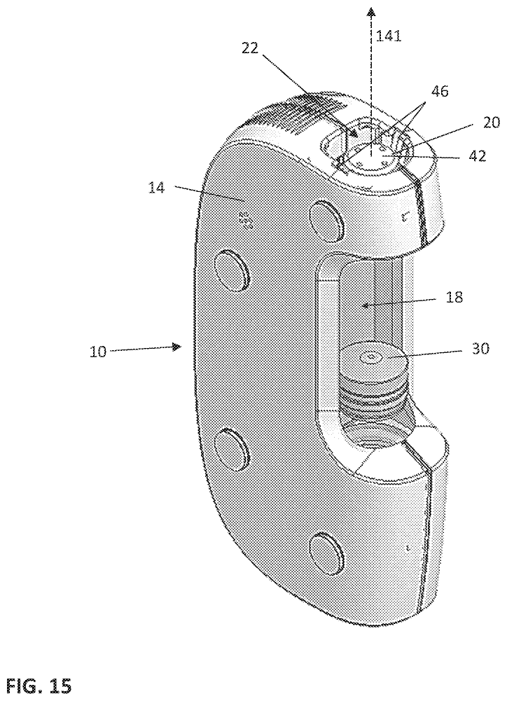

Referring to FIGS. 15-17, the infusion pump 10 is depicted separate from the infusion set 60. The housing 14 of the infusion pump 10 is configured to receive thereon, or therein, the infusion set 60. An opening 22 may be defined on the housing 14 that is configured to receive the infusion set 60 thereon and to allow for operable connection between the infusion set 60) and the infusion pump 10, and particularly, between the infusion set 60) and the outlet 20 of the reservoir 18. In some aspects, the opening 22 may define a recess, channel, guide, trough, or opening on, or through, the housing 14. It will be appreciated that the opening 22 should be sized and dimensioned such that the infusion set 60 can be appropriately received thereon or therein to allow for the desired operative connection between the infusion set 60 and the infusion pump 10.

In some aspects, the outlet 20 of the reservoir 18 may be sealed by a membrane or septum 42. The septum 42 may be arranged to cover part or all over the outlet 20, such that the reservoir 18 is sealed off from the opening 22 and is not in liquid communication with the opening 22. Inclusion of such a septum 42 may be advantageous in preventing debris, microorganisms, or other unwanted particulates from entering the reservoir 18. This can facilitate maintaining sterility of the reservoir 18 and the liquid therein. During use, the septum 42 may be removed or moved relative to the outlet 20, such that the septum 42 does not obstruct the outlet 20. When the septum 42 is removed, the reservoir 18 is in liquid communication with the opening 22. In some aspects, the septum 42 may be pierced by a suitable object, such as a needle (as will be explained in detail below), to place the outlet 20 and the reservoir 18 into liquid communication with the opening 22 and/or with one or more other components of the infusion pump 10 and/or infusion set 60. It should be understood that in such embodiments, the septum 42 should include a material that is configured to be pierced by the desired piercing component. In some aspects, the septum 42 may include a resealable material that is configured to be punctured by a piercing component to allow liquid to flow therethrough while the piercing component extends through the septum 42 and to seal around the piercing component during liquid flow and/or to seal the opening created by the piercing component when the piercing component is removed from the septum 42, thus precluding undesired leaks of liquid from the reservoir 18.

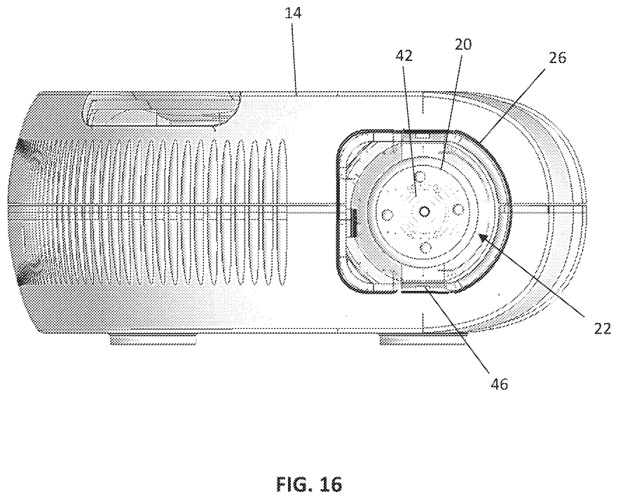

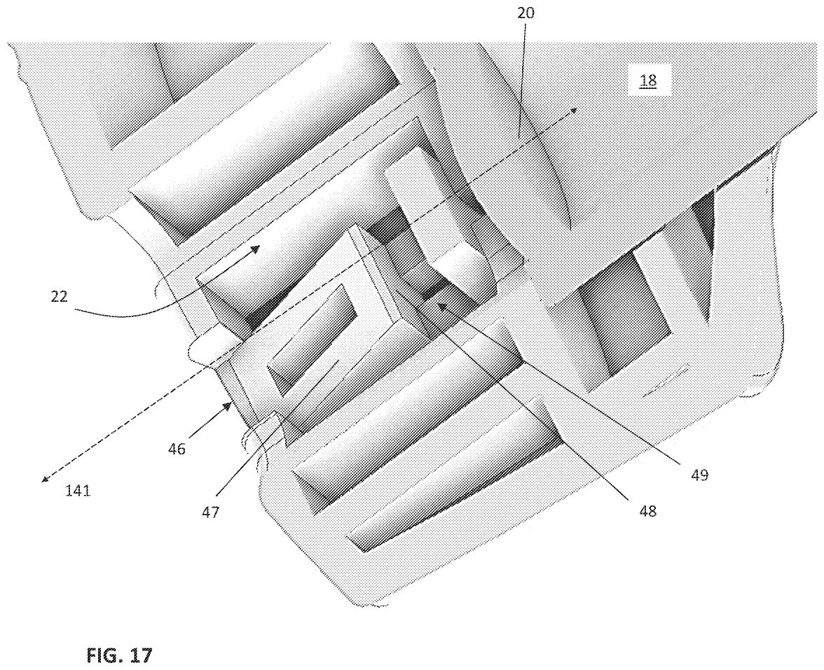

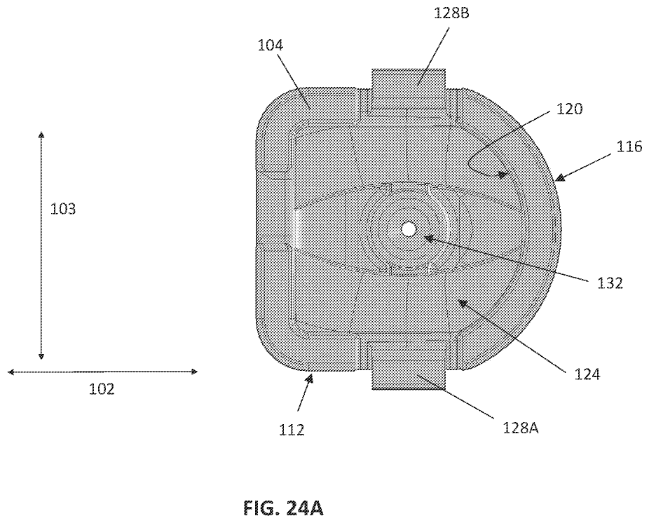

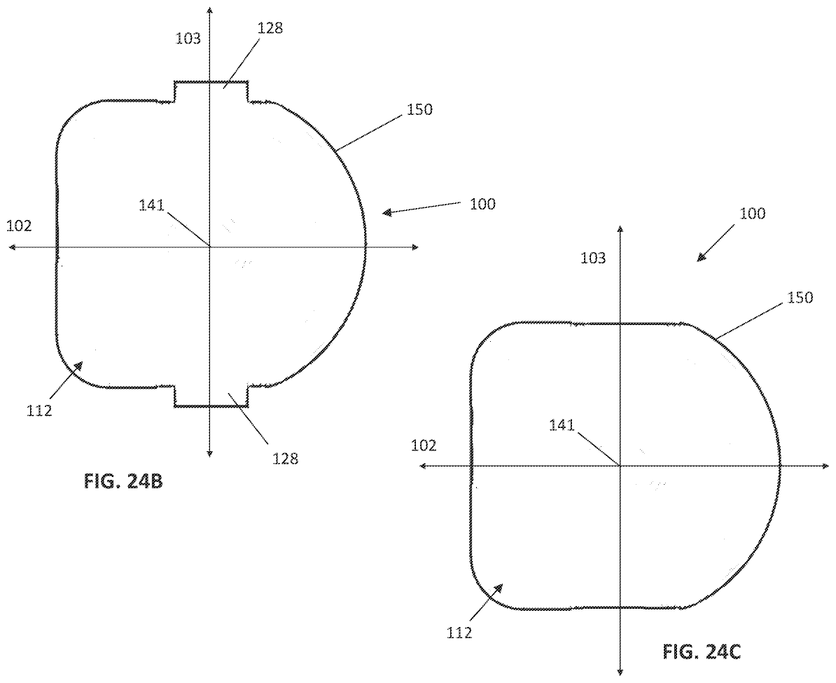

In some aspects, it may be preferable to connect the infusion set 60 to the infusion pump 10 in a particular orientation. This may be advantageous in that the user is less likely to connect the infusion set 60 to the infusion pump 10 incorrectly, thus adversely affecting parameters of the injection. As shown in FIG. 16, the opening 22 may include a specific cross-sectional shape 26 (shown as an outline relative to the opening 22) that is configured to correspond to a complementary cross-sectional shape 150 of the infusion set 60 (shown in FIGS. 24A-24C) as will be described in detail further below. Referring again to FIG. 15, the opening 22 may further include one or more retention members 46 configured to engage with the infusion set 60. Referring now to the cross-sectional view in FIG. 17, each retention member 46 may include a ramped surface 47 and a blocking surface 48. The components of the retention members 46 are configured to engage with corresponding components on the infusion set 60, as will be described in detail below.

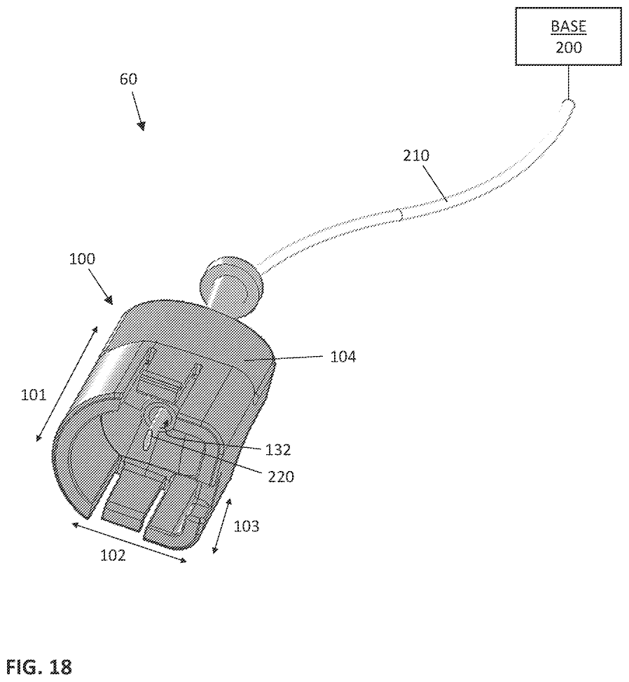

Turning to FIG. 18, an infusion set 60 is depicted having a connector 100, a base 200, and a tube or cannula 210 extending between the base 200 and the connector 100. The cannula 210 may be any suitable conduit configured to receive therein, and transfer therethrough, a liquid. The base 200 is configured to discharge the liquid from the cannula 210 to the target site, for example, to an injection site. In some aspects, the base 200 may include, or may be further connected to, an injection device (not shown), such as a hypodermic needle or another suitable sharp. The base 200 may further include a skin-contacting surface and an adhesive for attaching the skin-contacting surface to the target site. The specific injection component will depend on the target site, the medicament being injected, and/or on other medical preferences, and this disclosure is not limited to a particular injection component or to a particular base 200.

In some aspects, the infusion set 60 may include a needle 220 configured to pierce a pierceable septum 42. The needle 220 may define a channel therethrough configured to receive the liquid from the reservoir 18 after the needle 220 has pierced the septum 42. The needle 220) may be in liquid communication with the cannula 210, such that the liquid from the reservoir 18 is moved into and through the needle 220 and into the cannula 210. The particular dimensions of the needle 220 can depend on the liquid being dispensed from the infusion pump 10, and this disclosure is not limited to a particular needle size, shape, or type.

The infusion set 60 includes a connector 100 that is configured to attach to the housing 14 of the infusion pump 10. The engagement between the connector 100 and the housing 14 allows for operable connection between the infusion set 60 and the infusion pump 10. For purposes of this disclosure, reference will be made to a longitudinal direction 101, which can correspond to the linear direction along which the connector 100 is moved to connect to the infusion pump 10. Relative movement of the connector 100, or the infusion set 60) as a whole, with reference to the infusion pump 10 can be referred to as moving along the longitudinal direction 101 towards or away from the infusion pump 10, and specifically, towards or away from the opening 22 of the infusion pump 10. For further reference purposes, a transverse direction 102 is defined as being orthogonal to the longitudinal direction 101, and a lateral direction 103 is defined as being orthogonal to both the longitudinal direction 101 and the transverse direction 102. Thus, a plane defined by the longitudinal direction 101 and the transverse direction 102 will be orthogonal to the lateral direction 103: a plane defined by the longitudinal direction 101 and the lateral direction 103 will be orthogonal to the transverse direction 102; and a plane defined by the transverse direction 102 and the lateral direction 103 will be orthogonal to the longitudinal direction 101. It will be appreciated that the directions and orientations described above are for reference purposes only, and this disclosure is not limited to particular nomenclature of directions, planes, or axes. Unless otherwise specified herein, the terms “longitudinal,” “transverse,” and “lateral” are used to describe the orthogonal directional components of various components disclosed herein as designated by the longitudinal direction 101, transverse direction 102, and lateral direction 103.

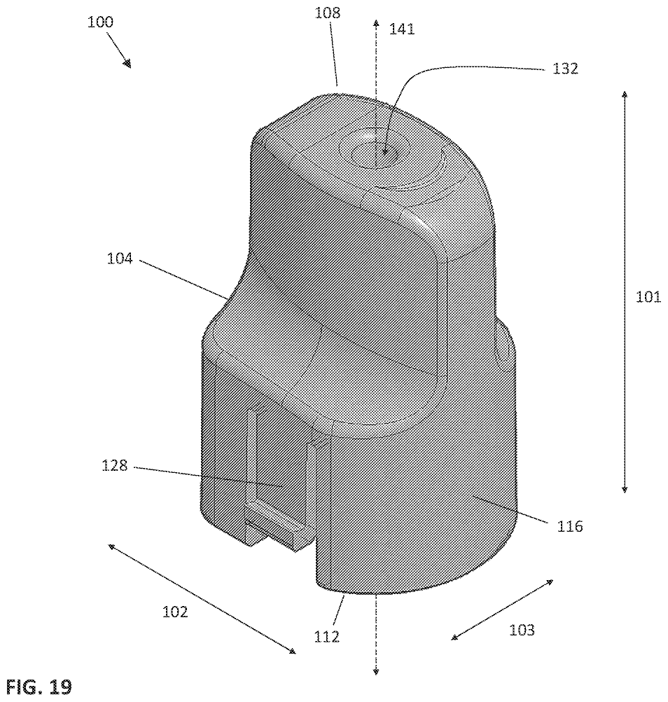

With continued reference to FIG. 18 and with further reference to FIGS. 19-23, an exemplary connector 100 is shown having a body 104 extending along the longitudinal direction 101 between a proximal end 108 and a distal end 112. The distal end 112 is spaced from the proximal end 108 along the longitudinal direction 101. For purposes of this disclosure, the terms “proximal” and “distal” generally refer to positions or directions toward and away from, respectively, the injection site to which the infusion set 60 can be connected. An insertion axis 141 is defined through the body 104. The insertion axis 141 extends in the direction from the proximal end 108 towards the distal end 112, or vice versa. The insertion axis 141 is parallel to the longitudinal direction 101 and is referenced for purposes of orienting the connector 100 relative to other components of the infusion set 60 and/or the infusion pump 10.

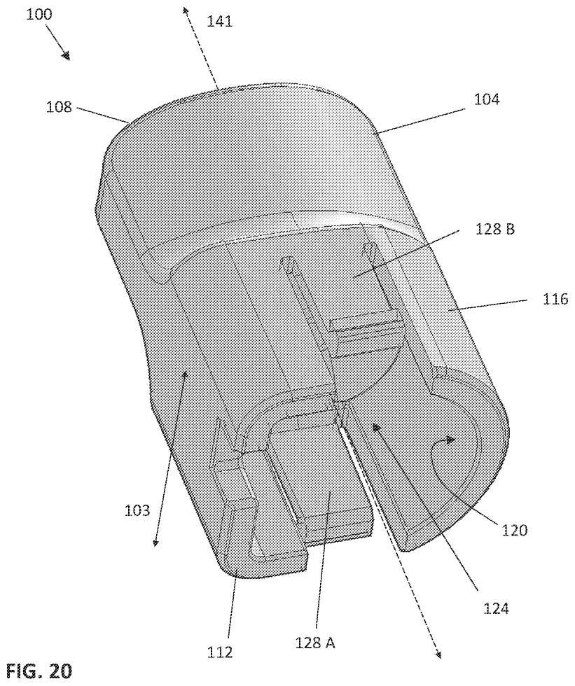

The body 104 includes an outer surface, or an outer wall, 116 and an inner surface, or an inner wall, 120 opposite the outer wall 116. The outer wall 116 is spaced from the inner wall 120 along a direction away from the insertion axis 141, for example, in a radial direction away from the insertion axis 141. As shown in FIG. 20, the inner wall 120 may define chamber or cavity 124 extending through the body 104 between the proximal end 108 and the distal end 112. The insertion axis 141 may extend through the cavity 124.

The body 104 may include a length measured along the longitudinal direction between the proximal end 108 and the distal end 112. The body 104 may include a width measured along the transverse direction 102 between a first point on the outer wall 116 and a second point on the outer wall 116 opposite the first point along the transverse direction 102. The body 104 may include a height measured along the lateral direction 103 between a third point on the outer wall 116 and a fourth point on the outer wall 116 opposite the third point along the lateral direction 103.

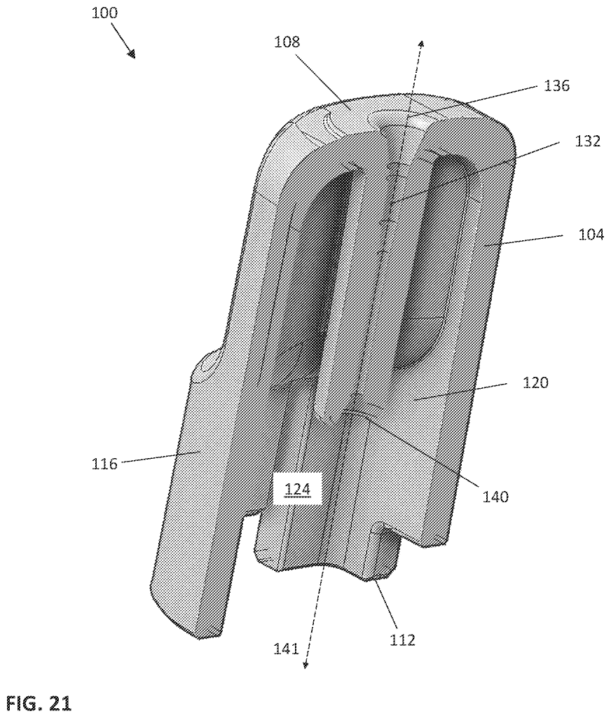

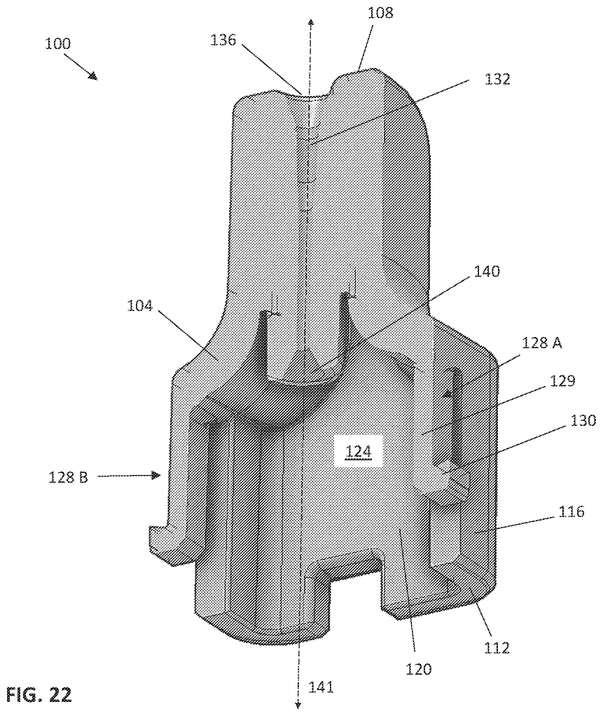

Referring to FIGS. 21 and 22, the body 104 may define a channel 132 extending through the body 104 between the proximal end 108 and the distal end 112. The channel 132 includes a proximal end 136 and a distal end 140 opposite the proximal end 136 and spaced from the proximal end 136 along the longitudinal direction 101. The insertion axis 141 may extend through the channel 132. The channel 132 may be arranged such that the insertion axis 141 extends through the entirety of the channel 132 through both the proximal and distal ends 136 and 140 of the channel 132. In some aspects, the channel 132 may be in fluid communication with the cavity 124. In some aspects, a portion of the channel 132 may be disposed within the cavity 124, for example, at least the distal end 140 of the channel 132 may be disposed within the cavity 124.

The channel 132 may define a cross-sectional dimension (e.g., a diameter) measured in the plane defined by the transverse direction 102 and the lateral direction 103. The cavity 124 may also define a cross-sectional dimension measured in the plane defined by the transverse and lateral directions 102 and 103. In some aspects, the cross-sectional dimension of the channel 132 may be smaller than the cross-sectional dimension of the cavity 124.

The channel 132 may include a length measured along the longitudinal direction 101 between the proximal end 136 and the distal end 140. In some aspects, the length of the channel 132 may be less than the length of the body 104. The channel 132 may be configured to receive the needle 220 therein. The needle 220 may be disposed partly within the channel 132, such that at least a portion of the needle 220 extends outside of the channel 132. In some aspects, the needle 220 may extend from the channel 132 past the distal end 140. In some aspects, the needle 220 may extend from the channel 132 past both, the distal end 140 and the proximal end 136. In some aspects, the cannula 210 may be disposed within the channel 132. The cannula 210 may be disposed into the channel 132 such that a portion of the cannula 210 extends out of the channel 132 at the proximal end 136.

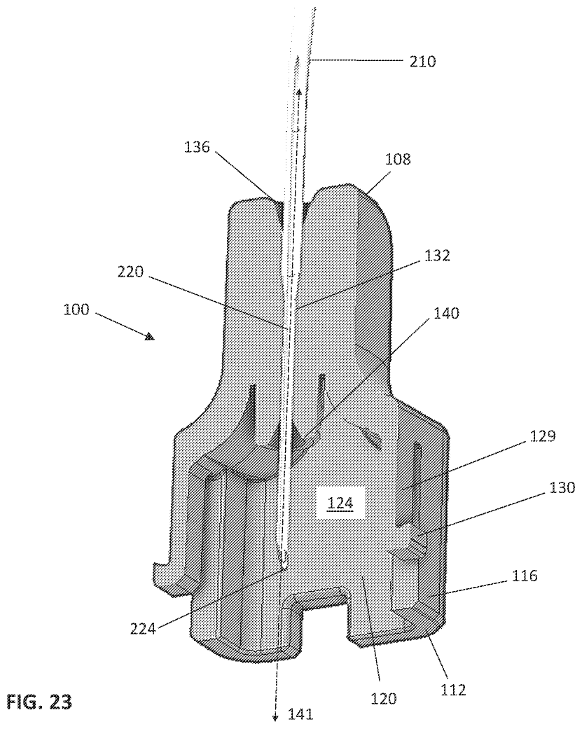

Referring to the exemplary aspect depicted in FIG. 23, the channel 132 may receive both the needle 220 and the cannula 210 therein. The needle 220 may be operably connected to the cannula 210. In some aspects, the needle 220 can be secured to the cannula 210 via an adhesive, such as an epoxy-based or acrylic-based adhesive. In some aspects, the adhesive can include ultraviolet glue (i.e., an adhesive that is cured by irradiation from ultraviolet light). In some aspects, the needle 220 can be mechanically retained relative to the cannula 210. For example, the needle 220 can be partially inserted into the cannula 210 to form a friction fit with the cannula 210. In some aspects, the needle 220 can be affixed to the cannula 210) via a combination of fixation mechanisms, such as a combination of friction fit and adhesive. In some aspects, the cannula 210 can be formed (e.g., via suitable molding techniques) to encompass the needle 220 therein.

As shown in FIG. 23, the needle 220 may be received partially within the channel 132 and may extend out of the channel 132 at the distal end 140, and the cannula 210, connected to the needle 220, may be received partially within the channel 132 and may extend out of the channel 132 at the proximal end 136. The insertion axis 141 may extend through the needle 220 and/or the cannula 210) when the needle 220 and/or the cannula 210) are disposed in the channel 132. The cannula 210 may extend out of and away from the connector 100. The needle 220 may be disposed, at least partially, in the cavity 124. The needle 220 may be secured within the channel 132 of the connector 100. In some aspects, the needle 220 can be affixed to the connector 100 via an adhesive, such as one or more of the adhesives described above. In some aspects, the connector 100 may be formed around the needle 220 such that the needle 220 partially defines the channel 132 of the connector 100. For example, in some aspects, the connector 100 may be formed by injection molding, and the needle 220 may be incorporated into the molding process such that the connector 100 is formed around the needle 220. The cannula 210 can be secured to the connector 100. In some aspects, the cannula 210 can be fixed to the connector 100 via an adhesive, such as described above. In some aspects, the cannula 210 may be part of the mold from which the connector 100 is formed during an injection molding process. In some aspects, the cannula 210 may be secured relative to the connector 100 by virtue of being fixedly attached to the needle 220, which can be secured to the connector 100.

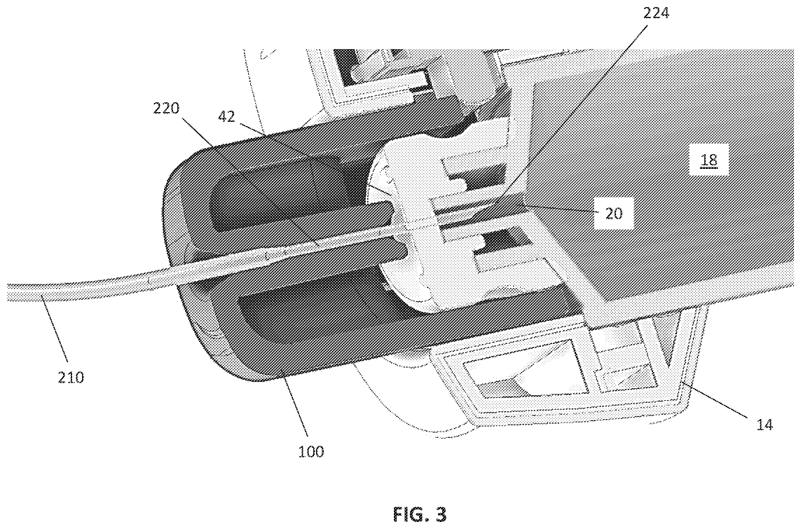

The needle 220 includes a needle tip 224 (labeled in FIGS. 3 and 23) configured for piercing. In some aspects, the needle 220 may be hollow and configured to receive a liquid therein at the needle tip 224. In some aspects, the hollow needle 220 may be configured to discharge liquid therefrom through the needle tip 224. The needle 220 may be disposed within the connector body 104 according to a desired position and/or orientation. The needle tip 224 may be positioned at a first distance measured from the proximal end 108 of the body 104 and the needle tip 224 along the longitudinal direction 101. In some aspects, the first distance may be less than the length of the connector body 104. The needle tip 224 may be disposed in the cavity 124. In some aspects, the first distance being less than the length of the connector body 104 may be advantageous to ensure that the needle tip 224 is within the cavity 124 and thus does not extend beyond the distal end 112 of the body 104. This can act as a safety feature to prevent a user from accidentally contacting the needle tip 224.

In some aspects, the connector 100 may be configured to be secured to the infusion pump 10, so that a user cannot inadvertently disconnect the infusion set 60 from the infusion pump 10. In some aspects, the infusion set 60 may be designed to be connected to, and to remain connected to, the infusion pump 10 after the desired use: that is, after use, the infusion set 60 can be precluded from being readily removed or separated from the infusion pump 10. This can be advantageous to prevent accidental injury of the user by the needle 220 in the infusion set 60. By preventing disconnection of the connector 100 from the infusion pump 10, the user does not have access to the needle 220 after initially connecting the infusion set 60 with the infusion pump 10. Such aspects may be further advantageous in that they decrease risk of inadvertently reusing components, such as the infusion set 60 and/or the infusion pump 10. One or more of the disclosed components may be designed to be single use, such that the user does not reuse these components. This decreases chances of infection or cross contamination that could be caused by utilizing previously used or improperly disposed components. Furthermore, the infusion pump 10 may include a predetermined volume of liquid medicament therein, and precluding re-use of the infusion pump 10 can help decrease risk of the user inadvertently using an infusion pump 10 that has already been used and receiving less medicament than desired.

The infusion set 60 may be connected to the infusion pump 10 via engagement between one or more respective retention members disposed on one or both of the infusion set 60 and the infusion pump 10. In some aspects, the connector 100 may include a retention member 128 thereon (shown in FIGS. 18-23) configured to engage with a respective retention member 46 on the infusion pump 10 (shown in FIGS. 15-27). The connector 100 may include a plurality of retention members 128, for example, 2, 3, . . . 8 or more retention members 128. It should be appreciated that the infusion pump 10 may include a complementary number of retention members 46, for example, to match the number of retention members 128 on the connector 100.

As visible in FIGS. 18-23, some aspects of the connector 100 may include two retention members 128. Each retention member 128 may be disposed on the body 104 and may extend, at least in part, from the outer wall 116. In some aspects, the two retention members 128 may be arranged approximately opposite each other along the transverse direction 102 or the lateral direction 103, but this disclosure is not limited to the particular arrangement of individual retention members 128. In the exemplary embodiment depicted in FIG. 20, the connector 100 is shown having a first retention member 128A arranged opposite a second retention member 128B that is spaced from the first retention member 128A along the lateral direction 103.

With specific reference to FIGS. 22 and 23, each retention member 128 may include an arm 129 extending from the body 104. The arm 129 may be connected to the body 104 at one end, such that the arm 129 is cantilevered relative to the body 104. The arm 129 may extend at least partly along the longitudinal direction 101. The arm 129 may be configured to be deflected away from the longitudinal direction 101. In some aspects, the arm 129 may be configured to be deflected towards the insertion axis 141 by application of a force onto the arm 129 in the same direction towards the insertion axis 141. When the application of the force is removed or decreased sufficiently, the arm 129 may return to its non-deflected position relative to the body 104. It will be understood that the arm 129 should include a resilient material that exhibits the desired elasticity parameters to allow the arm 129 to be deflected by an expected force and to substantially return to its non-deflected position when the force is removed or decreased. In some aspects, the arm 129 can include the same material that comprises the rest of the body 104.

A protrusion 130 extends from the arm 129. The protrusion 130 may, at least partially, extend from the arm 129 in a direction substantially perpendicular to the direction along the arm 129 extends from the body 104. That is, in some aspects, where the arm 129 extends along the longitudinal direction 101, the protrusion 130 may extend along the transverse direction 102 or the lateral direction 103. The protrusion 130 may extend at an angle of between about 0 and about 180 degrees, relative to the arm 129. In some aspects, the protrusion 130 may extend at an angle between about 45 and about 135 relative to the arm 129. In some aspects, the protrusion 130 may extend at about 90 degrees relative to the arm 129. Each arm 129 may include one or more protrusions 130 thereon. In some aspects, the force applied to the arm 129 to cause deflection of the arm 129 may be applied at the protrusion 130. The protrusion 130 may be configured to extend beyond the outer wall 116 of the body 104.

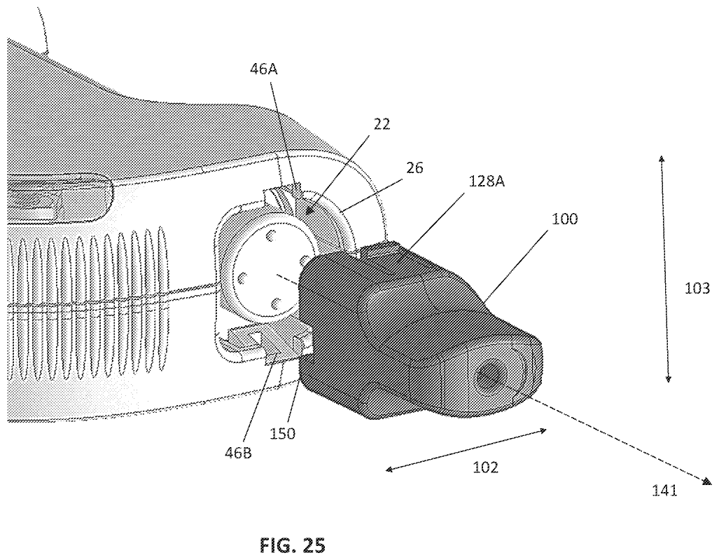

The infusion pump 10 may include corresponding retention members 46 configured to engage with the retention members 128 of the connector 100. The infusion pump 10 may include a complementary number of retention members 46 that are arranged to be engageable by the retention members 128. That is, the positioning of the retention members 46 on the infusion pump 10 may mirror the positioning of the retention members 128 on the connector 100. For example, as shown in the exemplary aspect of FIG. 25, the infusion pump 10 may have a first retention member 46A spaced from a second retention member 46B along the lateral direction 103. Upon connection of the infusion pump 10 to the infusion set 60 (connector 100 shown without rest of infusion set 60 in FIG. 25 for ease of reference), the first retention member 128A on the connector 100 is configured to engage with the first retention member 46A of the infusion pump 10. Similarly, the second retention member 128B of the connector 100 is configured to engage with the second retention member 46B of the infusion pump 10.

Referring to FIG. 17, an exemplary retention member 46 on the infusion pump 10 is depicted. The retention member 46 may include a sliding surface 47 and a blocking surface 48 adjacent the sliding surface 47. The blocking surface 48 partially defines a recess 49 adjacent to the sliding surface 47. The sliding surface 47 may be a ramp and may be inclined at an angle measured from the insertion axis 141. The blocking surface 48 may extend from the sliding surface 47. In operation, when the connector 100 is moved towards the infusion pump 10, the retention member 128 of the connector 100 contacts the sliding surface 47, moves along the sliding surface 47, and is placed in contact with, or adjacent to, the blocking surface 48 such that the connector 100 cannot be moved away from the infusion pump 10. The protrusion 130 on the connection member 128 contacts the sliding surface 47. The sliding surface 47 may be ramped towards the insertion axis 141. As the connector 100 continues to be moved along the insertion axis 141 towards the infusion pump 10, the protrusion 130 of a retention member 128 slides along the sliding surface 47 of the corresponding retention member 46. The contact of the sliding surface 47 with the protrusion 130 can exert a force onto the protrusion 130. It will be appreciated that the steeper the ramp incline of the sliding surface 47 is, the greater the rate of exerted force will be while the connector 100 is moved towards the infusion pump 10. The force acting on the protrusion 130 causes the arm 129 to deflect as described above. The arm 129 deflects towards the insertion axis 141. The deflection of the arm 129 biases the protrusion 130 away from the insertion axis 141. As the protrusion 130 passes the sliding surface 47, the protrusion 130 enters the recess 49 disposed adjacent the sliding surface 47. When the protrusion 130 is in the recess 49, the protrusion 130 does not contact the sliding surface 47. Because force exerted onto the protrusion 130 by the sliding surface 47 is removed when the protrusion 130 is in the recess 49, the arm 129 elastically returns to its substantially non-deformed position. This elastic movement by the arm 129 can create an audible noise, which can apprise a user that the infusion set 60 has been successfully connected to the infusion pump 10. The protrusion 130 is retained in the recess 49 adjacent to, or in contact with, the blocking surface 48. If the connector 100 is moved away from the infusion pump 10, for example, in an attempt to disconnect the infusion set 60 from the infusion pump 10, the protrusion 130 contacts the blocking surface 48 and is precluded from being moved past the blocking surface 48. This engagement between the retention member 128 and the retention member 46 allows for the infusion set 60 to be securely connected to the infusion pump 10 without risk of inadvertent disconnection.

The disclosed connection of the connector 100 with the housing 14 of the infusion pump 10 may be easier to form for the user than other types of connections. For example, when the connector 100 contacts the housing 14, the connector 100 can be moved linearly (along the insertion axis 141) relative to the infusion pump 10 without requiring further twisting or rotation of the connector 100 and/or the infusion pump 10 while resulting in the desired orientation and alignment of components. This may be preferable for users with limited or decreased mobility, eyesight, dexterity, and/or other motor functions that would hinder their completion of motions necessary for other non-linear types of connections.

The connector 100 may be dimensioned such that it can be connected with the infusion pump 10 in a preferred orientation. That is, the connector 100 may be designed to be connectable with the infusion pump 10 when the connector 100 is disposed in a first orientation relative to the infusion pump 10, and not connectable to the infusion pump 10 when the connector 100 is disposed in a second orientation relative to the infusion pump 10. This prevents accidental improper connection of the infusion set 60 with the infusion pump 10. For example, a user cannot connect the connector 100 upside down to the infusion pump 10, which can otherwise cause improper penetration of the septum 42 and inaccurate dispensing of the liquid in the reservoir 18. In some aspects, the particular design ensures that only the compatible infusion set 60 can be connected to an infusion pump 10, so as to reduce risk of the user accidentally connecting the infusion pump 10 to an incompatible infusion set 60.

Referring to FIG. 24A, the connector 100 is depicted at its distal end 112 shown in a plane defined by the transverse and lateral directions 102 and 103. The shape of the connector body 104 can be defined, for example, by the outer wall 116. The outer wall 116 can define the perimeter of the connector 100. With continued reference to FIG. 24A and with further reference to FIGS. 24B and 24C, exemplary cross-sectional shapes 150 of the connector 100 are depicted. The cross-sectional shape 150 can be defined by the outer wall 116 in the plane defined by the transverse and lateral directions 102 and 103. That is, the cross-sectional shape 150 can be defined in a plane that is orthogonal to the insertion axis 141. For purposes of this disclosure, the cross-sectional shape 150 can refer to a shape at, or adjacent to, the distal end 112 of the body 104. FIG. 24B depicts the cross-sectional shape 150 as including the outer wall 116 and the retention members 128. As described above, the retention members 128 are configured to be deflected inward towards the insertion axis 141. Thus, the retention members 128, when they are deflected, can be flush with the outer wall 116. A cross-sectional shape 150 depicting such an aspect is shown in FIG. 24C.

The cross-sectional shape 150 may define any suitable shape, such as circular, oblong, triangular, rectangular, pentagonal, hexagonal, or another suitable shape. In some aspects, the cross-sectional shape 150 may be substantially symmetrical along the transverse direction 102, the lateral direction 103, or both, when measured relative to the insertion axis 141. In some aspects, the cross-sectional shape 150 may be asymmetrical along the transverse direction 102, the lateral direction 103, or both, when measured relative to the insertion axis 141. In some exemplary aspects, the cross-sectional shape 150 may be substantially D-shaped, such as shown in FIG. 24C, for example. In some aspects, it may be advantageous for the cross-sectional shape to be asymmetrical with respect to at least one direction to allow for a keyed connection with the infusion pump 10.

The shape of the connector 100 can be configured to correspond to a respective shape of the opening 22 of the infusion pump housing 14. Referring to FIG. 16, an infusion pump 10 is shown having an exemplary cross-sectional shape 26 that is defined by the housing 14 and the opening 22. The cross-sectional shape 26 is shown being defined by the housing 14 and excluding the cutouts associated with the retention members 46. It should be understood that while the particular shapes of the cross-sectional shapes 150 and 26 are not limited by this disclosure, it may be beneficial to ensure that both cross-sectional shapes 150 and 26 complement each other, such that the connector 100 can be received into, or onto, the opening 22. As shown in FIG. 16, the cross-sectional shape 26 may be substantially D shaped to complement the cross-sectional shape 150 of the connector 100.

The connector 100, having the cross-sectional shape 150, can engage with the infusion pump 10, having the cross-sectional shape 26. Because the depicted cross-sectional shapes 150 and 26 are not fully symmetrical (at least along one of the transverse and lateral directions 102 and 103 with respect to the insertion axis 141), the connector 100 can be engaged with the opening 22 of the infusion pump 10 only when the connector 100 is oriented in the desired alignment and orientation relative to the infusion pump 10. That is, the cross-sectional shape 150 of the connector 100 should be oriented along the same direction as the cross-sectional shape 26 of the infusion pump 10 so that the connector 100 can be received in, or on, the opening 22. The cross-sectional shape 150 and the cross-sectional shape 26 should be disposed in planes that are parallel to each other. As shown in FIG. 25, for example, the connector 100 can be aligned relative to the infusion pump 10 along the insertion axis 141 such that one of the cross-sectional shapes 150 and 26 overlays the other of the cross-sectional shapes 150 and 26. If the connector 100 is in a different alignment, the cross-sectional shape 26 can be precluded from aligning with the cross-sectional shape 150, and thus the connector 100 cannot be connected with the opening 22 of the infusion pump 10. When the cross-sectional shape 150 of the connector 100 is substantially rotationally aligned with the cross-sectional shape 26 of the infusion pump 10, the one or more retention members 128 on the connector 100 can be rotationally aligned with the one or more complementary retention members 46 on the infusion pump 10.

In use, a user can connect the infusion set 60 to the infusion pump 10 by engaging the connector 100 with the opening 22 of the infusion pump 10. To connect the infusion set 60 to the infusion pump 10, the user can align the connector 100 relative to the infusion pump 10 along the insertion axis 141, as depicted, for example, in FIG. 25. The step of aligning the connector 100 may include translating the connector 100 along any one or more of the longitudinal direction 101, transverse direction 102, and lateral direction 103 relative to the infusion pump 10. The step of aligning the connector 100 may include rotating the connector 100 along any one or more of the longitudinal direction 101, transverse direction 102, and lateral direction 103 relative to the infusion pump 10. The connector 100 may be in the desired alignment and orientation when the cross-sectional shape 150 of the connector 100 substantially overlaps with the cross-sectional shape 26 of the opening 22 of the infusion pump 10 and when the cross-sectional shape 150 is in a plane defined by the transverse and lateral directions 102 and 103 that is parallel to a plane in which the cross-sectional shape 26 resides. It should be appreciated that the alignment of the cross-sectional shapes 150 and 26 will depend on the specific shape and size of the connector 100 and the infusion pump 10, as well as the desired engagement between the components. For example, in aspects where the opening 22 is configured to receive the connector 100 therein, the cross-sectional shape 150 of the connector 100 may be smaller than the cross-sectional shape 26 of the opening 22. In aspects where the opening 22 is configured to be received into the connector 100, the cross-sectional shape 150 may be larger than the cross-sectional shape 26.

After the connector 100 is aligned and oriented with the infusion pump 10, the connector 100 can be moved along the insertion axis 141 towards the infusion pump 10 so as to engage the opening 22. While it is described that the connector 100 is moving relative to the infusion pump 10, this disclosure is not limited to which component moves relative to the other, and the infusion pump 10 can be moved relative to the connector 100, or both the connector 100 and the infusion pump 10 can be moved towards each other.

Upon contacting the infusion pump 10, the connector 100 can continue to be moved along the insertion axis 141 towards the infusion pump 10. During this process, the one or more retention members 128 on the connector 100 can engage with the one or more respective retention members 46 on the infusion pump 10. As the connector 100 continues to be moved along the insertion axis 141, the protrusion 130 can contact the sliding surface 47 and slidably move along the sliding surface 47, deflecting the arm 129 during the process, until the protrusion 130 moves away from the sliding surface 47 and is disposed in the recess 49 (as described in detail above). At this time, the connector 100 can be fully engaged with the infusion pump 10. It should be further understood that, while the specific differences in dimensions between the cross-sectional shapes 150 and 26 are not limited by this disclosure, the tolerances between the connector 100 and the infusion pump 10 upon engagement should be such that the connector 100 is fixedly received in, or on, the infusion pump 10 so as to be substantially limited in its translation along one or more of the longitudinal direction 101, the transverse direction 102, and the lateral direction 103. Similarly, the connector 100 should be limited from rotation around any axes defined along one or more of the of the longitudinal direction 101, the transverse direction 102, and the lateral direction 103 relative to the infusion pump 10. When the connector 100 is fully engaged with the infusion pump 10, the connector 100 can contact one or more surfaces of the infusion pump 10 so as to be precluded from being further moved along the longitudinal direction 101 towards the infusion pump 10. In aspects where the connector 100 and the infusion pump 10 are configured to have engaging retention members 128 and 46 (as described above), when the connector 100 is fully engaged with the infusion pump 10, the connector 100 is further precluded from being moved along the longitudinal direction 101 away from the infusion pump 10.

In some aspects, it may be beneficial to limit or preclude some translation and/or rotation of the connector 100 relative to the infusion pump 10 during the connection process before the connector 100 is fully connected to and engaged with the opening 22. For example, the connector 100 may be partially engaged when a portion of the connector 100 is in contact with, or received into, the opening 22, but when the connector 100 is not fully engaged. When the connector 100 is partially engaged with the infusion pump 10, the connector 100 is permitted to be moved along the longitudinal direction 101 towards or away from the infusion pump 10, but the connector 100 is precluded from being translated along the transverse direction 102 and/or the lateral direction 103, and is further precluded from being rotated around the insertion axis 141 or around another axis extending in the longitudinal direction 101, transverse direction 102, and/or lateral direction 103.

During the step of moving the connector 100 towards the infusion pump 10, the needle 220 that can be affixed to the connector 100, is also being moved in the same direction as the connector 100. The needle 220 can be moved towards the outlet 20 of the reservoir 18. The needle tip 224 can be placed into contact with the septum 42. As the connector 100 continues to be moved towards the infusion pump 10, the needle tip 224 may puncture the septum 42. When the needle tip 224 passes the septum 42 along the longitudinal direction 101, the needle tip 224, and the needle 220 in general, is in liquid communication with the reservoir 18 of the infusion pump 10. At this time, liquid in the reservoir 18 may be moved into the infusion set 60 through the needle 220 by entering the needle 220 at the needle tip 224.

When the connector 100 is being moved towards the infusion pump 10, the cavity 124 of the connector 100 may be configured to receive the outlet 20 of the reservoir 18 therein. To the extent that the outlet 20 includes a septum 42 thereon, the cavity 124 is configured to receive the septum 42 therein. In some aspects, the cavity 124 may be configured to receive the outlet 20 and the septum 42 when the connector 100 is in the partially engaged position and when the connector 100 is in the fully engaged position.

When the connector 100 is securely fixed to the infusion pump 10, the user may actuate the infusion pump 10 to cause the liquid in the reservoir 18 to be moved therefrom and into the infusion set 60. The liquid can travel through the infusion set 60 towards the base 200, where the liquid can enter the user's body. It will be understood that other steps may be performed in addition to, or instead of, the steps described above. For example, additional steps may be performed associated with operation of the infusion pump 10, such as providing power to the infusion pump 10, turning on the suitable actuator (not shown), and/or controlling dispensing of the liquid from the infusion pump 10 to the infusion set 60. Additional steps may be performed to connect components to the infusion set 60, such as inserting the needle 220 into the connector 100, connecting the needle 220 and/or the connector 100 to the cannula 210, connecting the cannula 210 to the base 200, and/or connecting the base 200 to the user.

In some aspects, the infusion set 60 may be utilized with one particular infusion pump 10. In such cases, the infusion set 60 and the corresponding infusion pump 10 may form a pump assembly. In such aspects, the infusion set 60 may be configured to only operate with the desired complementary infusion pump 10. The infusion set 60 may include a connector 100 that is configured to engage with the desired infusion pump 10, but not with other infusion pumps 10. This distinction can be achieved by having different cross-sectional shapes 150 and 26 between different pump assemblies. For example, a connector 100 of a first pump assembly may have a cross-sectional shape 150 that corresponds to a cross-sectional shape 26 of an infusion pump 10 of the same first pump assembly, and a connector 100 of a second pump assembly may have a cross-sectional shape 150 that corresponds to a cross-sectional shape 26 of an infusion pump 10 of the same second pump assembly. However, the cross-sectional shape 150 of the connector 100 of the first pump assembly may not correspond to the cross-sectional shape 26 of the infusion pump 10 of the second pump assembly, thus precluding connection of the components. This may be advantageous to ensure that particular infusion sets 60 are connected only to the desired infusion pumps 10 and cannot be inadvertently connected to undesired infusion pumps 10, and vice versa.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

1-23. (canceled)

24. An infusion set configured to connect to an infusion pump having a housing containing a fluid reservoir, the infusion set comprising:
a connector having a body defining a channel at a proximal end of the body, the channel extending along a longitudinal axis, the body including:
an outer wall extending distally and defining at least a portion of an outer perimeter of the body, wherein the outer wall defines a wall cross-sectional shape along a plane perpendicular to the longitudinal axis, the cross-sectional shape configured to be asymmetrical along an axis perpendicular to the longitudinal axis and correspond with an opening cross-sectional shape of an opening of the housing, and
a body retention member configured to engage a housing retention member of the housing to retain the body with the housing;
a base configured to attach to an injection site; and
a flexible cannula extending from the proximal end of the body of the connector to the base and defining a fluid path from the connector to the base.

25. The infusion set of claim 24, wherein the wall cross-sectional shape is D-shaped.

26. The infusion set of claim 24, wherein, when the wall cross-sectional shape is substantially rotationally aligned with the opening cross-sectional shape, the body retention member is substantially rotationally aligned with the housing retention member.

27. The infusion set of claim 24, wherein the connector is rotationally fixed relative to the infusion pump when the connector is fully inserted within the opening of the housing.

28. The infusion set of claim 24, wherein the connector is rotationally fixed relative to the infusion pump when the connector is partially inserted within the opening of the housing.

29. The infusion set of claim 24, wherein the body further includes an inner wall spaced radially inward from the outer wall, wherein a distal end of the inner wall is positioned within the outer wall between the proximal end of the body and a distal end of the body opposite the proximal end.

30. The infusion set of claim 24, wherein the outer wall prevents rotation of the body of the connector when the connector is at least partially received within the opening of the housing.

31. The infusion set of claim 24, wherein the body retention member comprises a cantilevered arm configured to flex radially inward during insertion of the body through the opening of the housing and radially outward to engage the housing retention member to retain the body with the housing.

32. The infusion set of claim 24, wherein the body retention member is a first body retention member, the body further including a second body retention member configured to engage a second housing retention member of the housing to further retain the body with the housing.

33. The infusion set of claim 24, further comprising a needle positioned at least partially within the channel of the body, the needle extending distally from a distal end of the channel.

34. The infusion set of claim 24, wherein the channel extends about a channel axis, the channel axis being substantially parallel to a longitudinal axis of the body.

35. A pump assembly comprising:
an infusion set having a connector with a body that defines a channel at a proximal end of the body, the body including:
an outer wall extending distally and defining at least a portion of an outer perimeter of the body, wherein the outer wall defines a wall cross-sectional shape, and
a body retention member; and
a housing including a fluid reservoir, the housing defining an opening configured to receive at least a portion of the body of the connector, the housing including:
a housing retention member configured to engage the body retention member of the body to retain the body with the housing, wherein an opening cross-sectional shape of the opening of the housing corresponds with the wall cross-sectional shape of the outer wall of the body.

36. The pump assembly of claim 35, wherein the connector further includes a needle positioned at least partially within the channel of the body, the needle extending distally from a distal end of the channel.

37. The pump assembly of claim 36, wherein the fluid reservoir comprises a septum, the needle being configured to pierce the septum when the body is at least partially positioned within the opening of the housing.

38. The pump assembly of claim 37, wherein the outer wall of the body defines a cavity, wherein the septum is received within the cavity when the body is at least partially inserted into the opening of the housing.

39. The pump assembly of claim 36, wherein the housing further includes a plunger disposed within the fluid reservoir and a motor configured to drive the plunger through the fluid reservoir to drive fluid from the fluid reservoir through the needle of the connector.

40. The pump assembly of claim 35, wherein, when the wall cross-sectional shape is substantially rotationally aligned with the opening cross-sectional shape, the body retention member is substantially rotationally aligned with the housing retention member.

41. The pump assembly of claim 35, wherein the body retention member is a first retention member, and the housing retention member is a first housing retention member, wherein the body further includes a second retention member, and the housing further includes a second housing member configured to engage second body retention member of to further retain the body with the housing.

42. The pump assembly of claim 35, wherein engagement between the body retention member and the housing retention member irreversibly couples the connector to the housing.

43. The pump assembly of claim 35, wherein the connector is configured to be inserted into the opening along a linear trajectory such that the body of connector is rotationally immobile relative to the housing as it translates along the trajectory.
